(19) United States
(12) Patent Application Publication
(10) Pub. No.: US 2024/0298531 A1
(43) Pub. Date: Sep. 5, 2024

(54) HETEROCYCLIC COMPOUND AND ORGANIC LIGHT-EMITTING DEVICE INCLUDING SAME

(71) Applicant: LG CHEM, LTD., Seoul (KR)

(72) Inventors: Jae Tak LEE, Daejeon (KR); Jung Min YOON, Daejeon (KR); Heekyung YUN, Daejeon (KR); Su Jin HAN, Daejeon (KR); Dong Uk HEO, Daejeon (KR); Sung Kil HONG, Daejeon (KR)

(21) Appl. No.: 18/563,304

(22) PCT Filed: Mar. 16, 2023
(86) PCT No.: PCT/KR2023/003542

§ 371 Date: Nov. 21, 2023

(30) Foreign Application Priority Data
Mar. 21, 2022 (KR) 10-2022-0034756

(51) Int. Cl. H10K 85/60; C07D 403/10; C09K 11/06; H10K 50/16; H10K 50/17

(52) U.S. Cl. CPC H10K 85/654; C07D 403/10; C09K 11/06; H10K 85/615; C09K 2211/1018; H10K 50/16; H10K 50/171

(57) ABSTRACT

Provided is a heterocyclic compound of Chemical Formula 1:
[Chemical Formula 1]

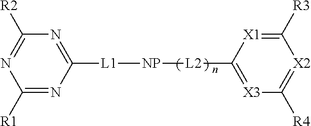

wherein: L1 is a direct bond or a substituted or unsubstituted arylene group; L2 is a substituted or unsubstituted arylene group; two or more of X1 to X3 are N and any remaining is CR′; Np is Chemical Formula 2:
[Chemical Formula 2]

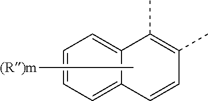

wherein: the dotted lines “------” are positions of attachment of Chemical Formula 2 where one is bonded to L1 and the other is bonded to L2 of Chemical Formula 1; and the other substituents are as defined in the specification; and an organic light-emitting device including the same. A light emitting device that includes the compound of Chemical Formula 1 in an organic material layer exhibits high efficiency and long service life.

### CROSS-REFERENCE TO RELATED APPLICATION(S)

This application is a National Stage Application of International Application No. PCT/KR2023/003542 filed on Mar. 16, 2023, which claims priority to and the benefit of Korean Patent Application No. 10-2022-0034756 filed in the Korean Intellectual Property Office on Mar. 21, 2022, the entire contents of which are incorporated herein by reference.

### TECHNICAL FIELD

The present specification relates to a hetero ring-containing compound and an organic light emitting device including the same.

### BACKGROUND ART

In general, an organic light emitting phenomenon refers to a phenomenon in which electric energy is converted into light energy by using an organic material. An organic light emitting device using the organic light emitting phenomenon usually has a structure including a positive electrode, a negative electrode, and an organic material layer interposed therebetween. Here, the organic material layer has in many cases a multi-layered structure composed of different materials in order to improve the efficiency and stability of the organic light emitting device, and for example, can be composed of a hole injection layer, a hole transport layer, a light emitting layer, an electron transport layer, an electron injection layer, and the like. In such a structure of the organic light emitting device, if a voltage is applied between the two electrodes, holes are injected from the positive electrode into the organic material layer and electrons are injected from the negative electrode into the organic material layer, and when the injected holes and electrons meet each other, an exciton is formed, and light is emitted when the exciton falls down again to a ground state.

There is a continuous need for developing a new material for the aforementioned organic light emitting device.

### CITATION LIST

Korean Patent Application Laid-Open No. 2003-012890

### BRIEF DESCRIPTION OF THE INVENTION

### Technical Problem

The present specification provides a hetero ring-containing compound and an organic light emitting device including the same.

### Technical Solution

An exemplary embodiment of the present specification provides a compound of the following Chemical Formula 1.

A compound of Chemical Formula 1:

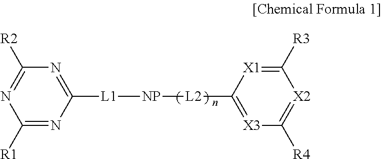

wherein in Chemical Formula 1:
L1 is a direct bond or a substituted or unsubstituted arylene group;
L2 is a substituted or unsubstituted arylene group;
n is 2 or 3, and the L2s are each the same as or different from each other;
X1 to X3 are the same as or different from each other, and are each independently N or CR′;
two or more of X1 to X3 are N;
R1 to R4 and R′ are the same as or different from each other, and are each independently hydrogen, deuterium, a halogen group, a substituted or unsubstituted alkyl group, a substituted or unsubstituted aryl group, or a substituted or unsubstituted heterocyclic group;
Np is represented by the following Chemical Formula 2:

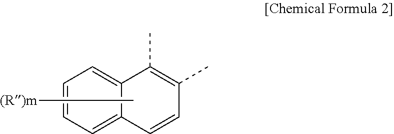

wherein in Chemical Formula 2:
the dotted lines “------” are positions of attachment of Chemical Formula 2 where one is bonded to L1 and the other is bonded to L2 of Chemical Formula 1:
R″ is hydrogen, deuterium, a halogen group, a substituted or unsubstituted alkyl group, a substituted or unsubstituted aryl group, or a substituted or unsubstituted heterocyclic group;
m is an integer from 0 to 6, and when m is 2 or higher, two or more R″s are the same as or different from each other.

Further, an exemplary embodiment of the present specification provides an organic light emitting device including: an anode; a cathode; and an organic material layer having one or more layers provided between the anode and the cathode, in which one or more layers of the organic material layer include the compound of Chemical Formula 1.

### Advantageous Effects

The compound described in the present specification can be used as a material for an organic material layer of an organic light emitting device. The compound according to at least one exemplary embodiment can improve the efficiency, achieve low driving voltage and/or improve lifetime characteristics in the organic light emitting device. In particular, the compound described in the present specification can be used as a material for hole injection, hole transport, hole injection and hole transport, electron blocking, light emission, hole blocking, electron transport, or electron injection. In addition, the organic light emitting device in which the compound described in the present specification is used has the effects of a low driving voltage, a high efficiency and/or a long service life compared to existing organic light emitting devices.

### BRIEF DESCRIPTION OF DRAWINGS

FIG. 1 illustrates an example of an organic light emitting device in which a substrate 1, an anode 2, an organic material layer 21, and a cathode 10 are sequentially stacked.

FIG. 2 illustrates an example of an organic light emitting device in which a substrate 1, an anode 2, a hole injection layer 3, a hole transport layer 4, an electron blocking layer 5, a light emitting layer 6, a hole blocking layer 7, an electron transport layer 8, an electron injection layer 9, and a cathode 10 are sequentially stacked.

FIG. 3 illustrates an example of an organic light emitting device in which a substrate 1, an anode 2, a hole injection layer 3, a hole transport layer 4, an electron blocking layer 5, a light emitting layer 6, a hole blocking layer 7, an electron injection and transport layer 11, and a cathode 10 are sequentially stacked.

### EXPLANATION OF REFERENCE NUMERALS AND SYMBOLS

1: Substrate
2: Anode
3: Hole injection layer
4: Hole transport layer
5: Electron blocking layer
6: Light emitting layer
7: Hole blocking layer
8: Electron transport layer
9: Electron injection layer
10: Cathode
11: Electron injection and transport layer
21: Organic material layer

### DETAILED DESCRIPTION

Hereinafter, the present specification will be described in more detail.

When one part “includes” one constituent element in the present specification, unless otherwise specifically described, this does not mean that another constituent element is excluded, but means that another constituent element can be further included.

When one member is disposed “on” another member in the present specification, this includes not only a case where the one member is brought into contact with another member, but also a case where still another member is present between the two members.

In the present specification, “------” or a dotted line means a position bonded to a chemical formula or a compound.

In the present specification, the deuterium substitution rate of a compound can be understood by a method of calculating the substitution rate based on the max. value of the distribution which molecular weights form at the end point of a reaction using thin-layer chromatography/mass spectrometry (TLC-MS) or a quantitative analysis method using NMR, and a method of adding DMF as an internal standard and calculating the D-substitution rate from the integrated amount of the total peak using the integration rate on 1H NMR.

In the present specification, “energy level” means a size of energy. Therefore, the energy level is interpreted to mean the absolute value of the corresponding energy value. For example, a low or deep energy level means that the absolute value increases in the negative direction from the vacuum level.

In the present specification, the highest occupied molecular orbital (HOMO) means a molecular orbital (highest occupied molecular orbital) in the highest energy region in regions in which electrons can participate in bonding, the lowest unoccupied molecular orbital (LUMO) means the molecular orbital (lowest unoccupied molecular orbital) in which electrons are present in the lowest energy region among the semi-bonded regions, and the HOMO energy level means the distance from the vacuum level to the HOMO. Furthermore, the LUMO energy level means the distance from the vacuum level to the LUMO.

In the present specification, a bandgap means a difference in energy level between HOMO and LUMO, that is, a HOMO-LUMO gap (Gap).

In the present specification, the HOMO energy level can be measured using a photoelectron spectrometer under the atmosphere (manufactured by RIKEN KEIKI Co., Ltd.: AC3), and the LUMO energy level can be calculated from wavelength values measured through photoluminescence (PL).

Examples of the substituents in the present specification will be described below, but are not limited thereto.

The term “substitution” means that a hydrogen atom bonded to a carbon atom of a compound is changed into another substituent, and a position to be substituted is not limited as long as the position is a position at which the hydrogen atom is substituted, that is, a position at which the substituent can be substituted, and when two or more are substituted, the two or more substituents can be the same as or different from each other.

In an exemplary embodiment of the present specification, the term “substituted or unsubstituted” means being substituted with one or two or more substituents selected from the group consisting of deuterium, a halogen group, a nitrile group (—CN), a nitro group, a hydroxyl group, an alkyl group, a cycloalkyl group, an alkoxy group, a phosphine oxide group, an aryloxy group, an alkylthioxy group, an arylthioxy group, an alkylsulfoxy group, an arylsulfoxy group, an alkenyl group, a silyl group, a boron group, an amine group, an aryl group, or a heterocyclic group, being substituted with a substituent in which two or more substituents among the exemplified substituents are linked, or having no substituent. For example, “the substituent in which two or more substituents are linked” can be a biphenyl group. That is, the biphenyl group can also be an aryl group, and can be interpreted as a substituent to which two phenyl groups are linked.

In an exemplary embodiment of the present specification, the term “substituted or unsubstituted” means being substituted with one or two or more substituents selected from the group consisting of deuterium, a halogen group, a nitrile group, a nitro group, a hydroxyl group, an amino group, a silyl group, a boron group, an alkoxy group, an aryloxy group, an alkyl group, a cycloalkyl group, an aryl group, and a heterocyclic group, being substituted with a substituent in which two or more substituents among the above-exemplified substituents are linked, or having no substituent.

In an exemplary embodiment of the present specification, the term “substituted or unsubstituted” means being substituted with one or two or more substituents selected from the group consisting of deuterium, an alkyl group, an aryl group, and a heterocyclic group, being substituted with a substituent in which two or more substituents among the exemplified substituents are linked, or having no substituent.

In the present specification, N % substitution with deuterium means that N % of hydrogen available in the corresponding structure is substituted with deuterium. For example, 25% substitution of dibenzofuran with deuterium means that two of eight hydrogens of dibenzofuran are substituted with deuteriums.

Examples of the substituents will be described below, but are not limited thereto.

In the present specification, examples of a halogen group include a fluoro group (—F), a chloro group (—Cl), a bromo group (—Br), or an iodo group (—I).

In the present specification, a silyl group can be a chemical formula of —SiYₐY_{b}Y_{c}, and the Yₐ, Y_{b}, and Y_{c} can be each hydrogen, a substituted or unsubstituted alkyl group, or a substituted or unsubstituted aryl group. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a boron group can be a chemical formula of —BY_{d}Yₑ, and the Y_{d} and Yₑ can be each hydrogen, a substituted or unsubstituted alkyl group, or a substituted or unsubstituted aryl group. Specific examples of the boron group include a trimethylboron group, a triethylboron group, a t-butyldimethylboron group, a triphenylboron group, a phenylboron group, and the like, but are not limited thereto.

In the present specification, the alkyl group can be straight-chained or branched, and the number of carbon atoms thereof is not particularly limited, but is preferably 1 to 60. According to an exemplary embodiment, the number of carbon atoms of the alkyl group is 1 to 30. According to another exemplary embodiment, the number of carbon atoms of the alkyl group is 1 to 20. According to still another exemplary embodiment, the number of carbon atoms of the alkyl group is 1 to 10. Specific examples of the alkyl group include a methyl group, an ethyl group, a propyl group, an n-propyl group, an isopropyl group, a butyl group, an n-butyl group, an isobutyl group, a tert-butyl group, a pentyl group, an n-pentyl group, a hexyl group, an n-hexyl group, a heptyl group, an n-heptyl group, an octyl group, an n-octyl group, and the like, but are not limited thereto.

In the present specification, the above-described description on the alkyl group can be applied to an arylalkyl group, except that the arylalkyl group is substituted with an aryl group.

In the present specification, the alkoxy group can be straight-chained, branched, or cyclic. The number of carbon atoms of the alkoxy group is not particularly limited, but is preferably 1 to 20. Specific examples thereof include methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, and the like, but are not limited thereto.

Substituents including an alkyl group, an alkoxy group, and other alkyl group moieties described in the present specification include both a straight-chained form and a branched form.

In the present specification, an alkenyl group can be straight-chained or branched, and the number of carbon atoms thereof is not particularly limited, but is preferably 2 to 40. According to an exemplary embodiment, the number of carbon atoms of the alkenyl group is 2 to 20. According to another exemplary embodiment, the number of carbon atoms of the alkenyl group is 2 to 10. According to still another exemplary embodiment, the number of carbon atoms of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present specification, the alkynyl group can be straight-chained or branched as a substituent including a triple bond between a carbon atom and a carbon atom, and the number of carbon atoms thereof is not particularly limited, but is preferably 2 to 40. According to an exemplary embodiment, the number of carbon atoms of the alkynyl group is 2 to 20. According to another exemplary embodiment, the number of carbon atoms of the alkynyl group is 2 to 10.

In the present specification, a cycloalkyl group is not particularly limited, but has preferably 3 to 60 carbon atoms, and according to an exemplary embodiment, the number of carbon atoms of the cycloalkyl group is 3 to 30. According to another exemplary embodiment, the number of carbon atoms of the cycloalkyl group is 3 to 20. According to still another exemplary embodiment, the number of carbon atoms of the cycloalkyl group is 3 to 6. Specific examples thereof include a cyclopropyl group, a cyclobutyl group, a cyclopentyl group, a cyclohexyl group, a cycloheptyl group, a cyclooctyl group, an adamantyl group, and the like, but are not limited thereto.

In the present specification, an amine group is —NH₂, and the amine group can be substituted with the above-described alkyl group, aryl group, heterocyclic group, alkenyl group, cycloalkyl group, a combination thereof, and the like. The number of carbon atoms of the substituted amine group is not particularly limited, but is preferably 1 to 30. According to an exemplary embodiment, the number of carbon atoms of the amine group is 1 to 20. According to an exemplary embodiment, the number of carbon atoms of the amine group is 1 to 10. Specific examples of the substituted amine group include a methylamine group, a dimethylamine group, an ethylamine group, a diethylamine group, a phenylamine group, a 9,9-dimethylfluorenylphenylamine group, a pyridylphenylamine group, a diphenylamine group, a phenylpyridylamine group, a naphthylamine group, a biphenylamine group, an anthracenylamine group, a dibenzofuranylphenylamine group, a 9-methylanthracenylamine group, a diphenylamine group, a phenylnaphthylamine group, a ditolylamine group, a phenyltolylamine group, a diphenylamine group, and the like, but are not limited thereto.

In the present specification, an aryl group is not particularly limited, but has preferably 6 to 60 carbon atoms, and can be a monocyclic aryl group or a polycyclic aryl group. According to an exemplary embodiment, the number of carbon atoms of the aryl group is 6 to 30. According to an exemplary embodiment, the number of carbon atoms of the aryl group is 6 to 20. Examples of the monocyclic aryl group include a phenyl group, a biphenyl group, a terphenyl group, a quaterphenyl group, and the like, but are not limited thereto. Examples of the polycyclic aryl group include a naphthyl group, an anthracenyl group, a phenanthrenyl group, a pyrenyl group, a perylenyl group, a triphenyl group, a chrysenyl group, a fluorenyl group, a triphenylenyl group, and the like, but are not limited thereto.

In the present specification, the substituted aryl group can include a structure in which an aliphatic hydrocarbon ring is fused to the aryl group. According to an exemplary embodiment, the substituted aryl group can include a tetrahydronaphthalene group, and further specifically, can include

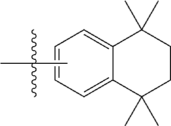

(a 1,1,4,4-tetramethyl-1,2,3,4-tetrahydronaphthalene group), but is not limited thereto.

In the present specification, a fluorenyl group can be substituted, and two substituents can be bonded to each other to form a spiro structure. In this case, the spiro structure can be an aromatic hydrocarbon ring or an aliphatic hydrocarbon ring.

When the fluorenyl group is substituted, the substituent can be a spirofluorenyl group such as

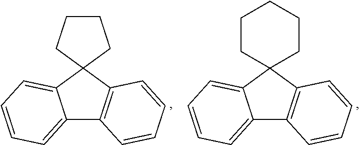

and

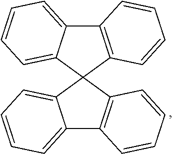 [SMILES: c1ccc2c(c1)-c1ccccc1C21c2ccccc2-c2ccccc21]

and a substituted fluorenyl group such as

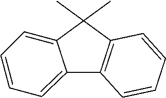

(a 9, 9-dimethylfluorenyl group), and

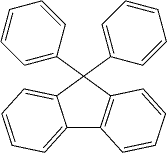 [SMILES: c1ccc(C2(c3ccccc3)c3ccccc3-c3ccccc32)cc1]

(a 9,9-diphenylfluorenyl group). However, the substituent is not limited thereto.

In the present specification, the above-described description on the aryl group can be applied to an aryl group in an aryloxy group.

In the present specification, the above-described description on the alkyl group can be applied to an alkyl group in the alkylthioxy group and the alkylsulfoxy group.

In the present specification, the above-described description on the aryl group can be applied to an aryl group in the arylthioxy group and the arylsulfoxy group.

In the present specification, a heterocyclic group is a cyclic group including one or more of N, O, P, S, Si, and Se as a heteroatom, and the number of carbon atoms thereof is not particularly limited, but is preferably 2 to 60. According to an exemplary embodiment, the number of carbon atoms of the heterocyclic group is 2 to 30. According to an exemplary embodiment, the number of carbon atoms of the heterocyclic group is 2 to 20. Examples of the heterocyclic group include a pyridine group, a pyrrole group, a pyrimidine group, a quinoline group, a pyridazinyl group, a furan group, a thiophene group, an imidazole group, a pyrazole group, a dibenzofuran group, a dibenzothiophene group, a carbazole group, a benzocarbazole group, a naphthobenzofuran group, a benzonaphthothiophene group, an indenocarbazole group, a triazinyl group, and the like, but are not limited thereto.

In the present specification, the above-described description on the heterocyclic group can be applied to a heteroaryl group except for an aromatic heteroaryl group.

In the present specification, the description on the aryl group can be applied to an arylene group except for a divalent arylene group.

In the present specification, the description on the heterocyclic group can be applied to a divalent hetero ring except for a divalent hetero ring.

In the present specification, in a substituted or unsubstituted ring formed by bonding adjacent groups, the “ring” means a hydrocarbon ring; or a hetero ring.

The hydrocarbon ring can be an aromatic ring, an aliphatic ring, or a fused ring of the aromatic ring and the aliphatic ring, and can be selected from the examples of the cycloalkyl group or the aryl group.

In the present specification, being bonded to an adjacent group to form a ring means being bonded to an adjacent group to form a substituted or unsubstituted aliphatic hydrocarbon ring; a substituted or unsubstituted aromatic hydrocarbon ring; a substituted or unsubstituted aliphatic hetero ring; a substituted or unsubstituted aromatic hetero ring; or a fused ring thereof. The hydrocarbon ring means a ring composed only of carbon and hydrogen atoms. The hetero ring means a ring including one or more selected from elements such as N, O, P, S, Si and Se. In the present specification, the aliphatic hydrocarbon ring, the aromatic hydrocarbon ring, the aliphatic hetero ring, and the aromatic hetero ring can be monocyclic or polycyclic.

In the present specification, the aliphatic hydrocarbon ring means a ring composed only of carbon and hydrogen atoms as a non-aromatic ring. Examples of the aliphatic hydrocarbon ring include cyclopropane, cyclobutane, cyclobutene, cyclopentane, cyclopentene, cyclohexane, cyclohexene, 1,4-cyclohexadiene, cycloheptane, cycloheptene, cyclooctane, cyclooctene, and the like, but are not limited thereto.

In the present specification, an aromatic hydrocarbon ring means an aromatic ring composed only of carbon and hydrogen atoms. Examples of the aromatic hydrocarbon ring include benzene, naphthalene, anthracene, phenanthrene, perylene, fluoranthene, triphenylene, phenalene, pyrene, tetracene, chrysene, pentacene, fluorene, indene, acenaphthylene, benzofluorene, spirofluorene, and the like, but are not limited thereto. In the present specification, the aromatic hydrocarbon ring can be interpreted to have the same meaning as the aryl group.

In the present specification, an aliphatic hetero ring means an aliphatic ring including one or more of hetero atoms. Examples of the aliphatic hetero ring include oxirane, tetrahydrofuran, 1,4-dioxane, pyrrolidine, piperidine, morpholine, oxepane, azocane, thiocane, and the like, but are not limited thereto.

In the present specification, an aromatic hetero ring means an aromatic ring including one or more of hetero atoms. Examples of the aromatic hetero ring include pyridine, pyrrole, pyrimidine, pyridazine, furan, thiophene, imidazole, pyrazole, oxazole, isoxazole, thiazole, isothiazole, triazole, oxadiazole, thiadiazole, dithiazole, tetrazole, pyran, thiopyran, diazine, oxazine, thiazine, dioxine, triazine, tetrazine, isoquinoline, quinoline, quinone, quinazoline, quinoxaline, naphthyridine, acridine, phenanthridine, diaza naphthalene, triazaindene, indole, indolizine, benzothiazole, benzoxazole, benzoimidazole, benzothiophene, benzofuran, dibenzothiophene, dibenzofuran, carbazole, benzocarbazole, dibenzocarbazole, phenazine, imidazopyridine, phenoxazine, indolocarbazole, indenocarbazole, and the like, but are not limited thereto.

Hereinafter, preferred exemplary embodiments of the present invention will be described in detail. However, the exemplary embodiments of the present invention can be modified into various other forms, and the scope of the present invention is not limited to the exemplary embodiments which will be described below.

The compound of Chemical Formula 1 according to the present invention shows the effects of improving efficiency by including triazine and/or pyrimidine in naphthalene in the form of a dimer to increase electron mobility, and increasing the service life of the organic light emitting device by setting the linker length of triazine to 1 or less and setting the length of the other N-containing ring group to 2 or more linkers to regulate the electron injection characteristics.

Additionally, when the compound of Chemical Formula 1 of the present invention includes deuterium, the efficiency and service life of the device are improved. Specifically, when hydrogen is replaced with deuterium, the chemical properties of the compound hardly change, but the physical properties of the deuterated compound change, so that the vibration energy level is lowered. The compound substituted with deuterium can prevent a decrease in quantum efficiency caused by a decrease in intermolecular Van der Waals force or a collision due to intermolecular vibration. Further, the C-D bond can improve stability of a compound.

Therefore, when the above-described compound of Chemical Formula 1 is applied to an organic light emitting device, it is possible to obtain an organic light emitting device having high efficiency, low voltage, and/or long service life characteristics.

Hereinafter, Chemical Formula 1 will be described in detail.

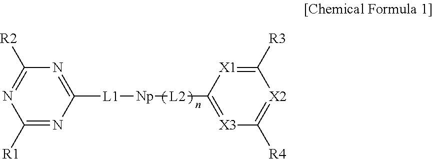

In Chemical Formula 1:
L1 is a direct bond or a substituted or unsubstituted arylene group;
L2 is a substituted or unsubstituted arylene group,
n is 2 or 3, and the L2s are each the same as or different from each other;
X1 to X3 are the same as or different from each other, and are each independently N; or CR′;
two or more of X1 to X3 are N;
R1 to R4 and R′ are the same as or different from each other, and are each independently hydrogen; deuterium, a halogen group, a substituted or unsubstituted alkyl group, a substituted or unsubstituted aryl group, or a substituted or unsubstituted heterocyclic group;
Np is represented by the following Chemical Formula 2:

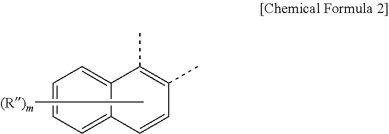

wherein in Chemical Formula 2:
the dotted lines “------” are positions of attachment of Chemical Formula 2 where one is bonded to L1 and the other is bonded to L2 of Chemical Formula 1;
R″ is hydrogen, deuterium, a halogen group, a substituted or unsubstituted alkyl group, a substituted or unsubstituted aryl group, or a substituted or unsubstituted heterocyclic group; and
m is an integer from 0 to 6, and when m is 2 or higher, the two or more R′s are the same as or different from each other.

In an exemplary embodiment of the present specification, L1 is a direct bond or a substituted or unsubstituted arylene group.

In an exemplary embodiment of the present specification, L1 is a direct bond; or a substituted or unsubstituted arylene group having 6 to 60 carbon atoms.

In an exemplary embodiment of the present specification, L1 is a direct bond or a substituted or unsubstituted arylene group having 6 to 30 carbon atoms.

In an exemplary embodiment of the present specification, L1 is a direct bond; or a substituted or unsubstituted arylene group having 6 to 20 carbon atoms.

In an exemplary embodiment of the present specification, L1 is a direct bond or a substituted or unsubstituted arylene group having 6 to 12 carbon atoms.

In an exemplary embodiment of the present specification, L1 is a direct bond or an arylene group which is unsubstituted or substituted with deuterium.

In an exemplary embodiment of the present specification, L1 is a direct bond or an arylene group having 6 to 60 carbon atoms, which is unsubstituted or substituted with deuterium.

In an exemplary embodiment of the present specification, L1 is a direct bond or an arylene group having 6 to 30 carbon atoms, which is unsubstituted or substituted with deuterium.

In an exemplary embodiment of the present specification, L1 is a direct bond or an arylene group having 6 to 20 carbon atoms, which is unsubstituted or substituted with deuterium.

In an exemplary embodiment of the present specification, L1 is a direct bond or an arylene group having 6 to 12 carbon atoms, which is unsubstituted or substituted with deuterium.

In an exemplary embodiment of the present specification, L1 is a direct bond, a substituted or unsubstituted phenylene group, a substituted or unsubstituted biphenylylene group, a substituted or unsubstituted terphenylylene group, a substituted or unsubstituted naphthylene group, a substituted or unsubstituted fluorenylene group, a substituted or unsubstituted phenanthrenylene group, or a substituted or unsubstituted triphenylenylylene group.

In an exemplary embodiment of the present specification, L1 is a direct bond, a substituted or unsubstituted phenylene group, a substituted or unsubstituted biphenylylene group, a substituted or unsubstituted terphenylylene group, a substituted or unsubstituted naphthylene group, or a substituted or unsubstituted fluorenylene group.

In an exemplary embodiment of the present specification, L1 is a direct bond, a substituted or unsubstituted phenylene group, a substituted or unsubstituted biphenylylene group, a substituted or unsubstituted terphenylylene group, or a substituted or unsubstituted naphthylene group.

In an exemplary embodiment of the present specification, L1 is a direct bond, a substituted or unsubstituted phenylene group, a substituted or unsubstituted biphenylylene group, or a substituted or unsubstituted naphthylene group.

In an exemplary embodiment of the present specification, L1 is a direct bond; a phenylene group that is unsubstituted or substituted with deuterium; a biphenylylene group that is unsubstituted or substituted with deuterium; or a naphthylene group that is unsubstituted or substituted with deuterium.

In an exemplary embodiment of the present specification, L1 is a direct bond, a phenylene group, a biphenylylene group, or a naphthylene group.

In an exemplary embodiment of the present specification, L1 is a direct bond, a phenylene group, or a biphenylylene group.

In an exemplary embodiment of the present specification, L1 is a direct bond or a substituted or unsubstituted phenylene group.

In an exemplary embodiment of the present specification, L1 is a direct bond; or a phenylene group that is unsubstituted or substituted with deuterium.

In an exemplary embodiment of the present specification, L1 is a direct bond or a phenylene group.

In an exemplary embodiment of the present specification, L1 is a direct bond.

In an exemplary embodiment of the present specification, L1 is a phenylene group.

In an exemplary embodiment of the present specification, L2 is a substituted or unsubstituted arylene group.

In an exemplary embodiment of the present specification, L2 is a substituted or unsubstituted arylene group having 6 to 60 carbon atoms.

In an exemplary embodiment of the present specification, L2 is a substituted or unsubstituted arylene group having 6 to 30 carbon atoms.

In an exemplary embodiment of the present specification, L2 is a substituted or unsubstituted arylene group having 6 to 20 carbon atoms.

In an exemplary embodiment of the present specification, L2 is a substituted or unsubstituted arylene group having 6 to 12 carbon atoms.

In an exemplary embodiment of the present specification, L2 is an arylene group which is unsubstituted or substituted with deuterium.

In an exemplary embodiment of the present specification, L2 is an arylene group having 6 to 60 carbon atoms, which is unsubstituted or substituted with deuterium.

In an exemplary embodiment of the present specification, L2 is an arylene group having 6 to 30 carbon atoms, which is unsubstituted or substituted with deuterium.

In an exemplary embodiment of the present specification, L2 is an arylene group having 6 to 20 carbon atoms, which is unsubstituted or substituted with deuterium.

In an exemplary embodiment of the present specification, L2 is an arylene group having 6 to 12 carbon atoms, which is unsubstituted or substituted with deuterium.

In an exemplary embodiment of the present specification, L2 is a substituted or unsubstituted phenylene group, a substituted or unsubstituted biphenylylene group, a substituted or unsubstituted terphenylylene group, a substituted or unsubstituted naphthylene group, a substituted or unsubstituted fluorenylene group, a substituted or unsubstituted phenanthrenylene group, or a substituted or unsubstituted triphenylenylylene group.

In an exemplary embodiment of the present specification, L2 is a substituted or unsubstituted phenylene group, a substituted or unsubstituted biphenylylene group, a substituted or unsubstituted terphenylylene group, a substituted or unsubstituted naphthylene group, or a substituted or unsubstituted fluorenylene group.

In an exemplary embodiment of the present specification, L2 is a substituted or unsubstituted phenylene group, a substituted or unsubstituted biphenylylene group, a substituted or unsubstituted terphenylylene group, or a substituted or unsubstituted naphthylene group.

In an exemplary embodiment of the present specification, L2 is a substituted or unsubstituted phenylene group, a substituted or unsubstituted biphenylylene group, or a substituted or unsubstituted naphthylene group.

In an exemplary embodiment of the present specification, L2 is a phenylene group that is unsubstituted or substituted with deuterium; a biphenylylene group that is unsubstituted or substituted with deuterium; or a naphthylene group that is unsubstituted or substituted with deuterium.

In an exemplary embodiment of the present specification, L2 is a phenylene group, a biphenylylene group, or a naphthylene group.

In an exemplary embodiment of the present specification, L2 is a phenylene group or a biphenylylene group.

In an exemplary embodiment of the present specification, L2 is a substituted or unsubstituted phenylene group.

In an exemplary embodiment of the present specification, L2 is a phenylene group which is unsubstituted or substituted with deuterium.

In an exemplary embodiment of the present specification, L2 is a phenylene group.

In an exemplary embodiment of the present specification, n is 2 or 3.

In an exemplary embodiment of the present specification, n is 2.

In an exemplary embodiment of the present specification, n is 3.

In an exemplary embodiment of the present specification, X1 to X3 are the same as or different from each other, and are each independently N or CR′

In an exemplary embodiment of the present specification, two or more of X1 to X3 are N.

In an exemplary embodiment of the present specification, two of X1 to X3 are N.

In an exemplary embodiment of the present specification, X1 and X2 are N.

In an exemplary embodiment of the present specification, X1 and X3 are N.

In an exemplary embodiment of the present specification, X2 and X3 are N.

In an exemplary embodiment of the present specification, all of X1 to X3 are N.

In an exemplary embodiment of the present specification, R1 to R4 and R′ are the same as or different from each other, and are each independently hydrogen, deuterium, a halogen group, a substituted or unsubstituted alkyl group, a substituted or unsubstituted aryl group, or a substituted or unsubstituted heterocyclic group.

In an exemplary embodiment of the present specification, R1 to R4 are the same as or different from each other, and are each independently hydrogen, deuterium, a halogen group, a substituted or unsubstituted alkyl group, a substituted or unsubstituted aryl group, or a substituted or unsubstituted heterocyclic group.

In an exemplary embodiment of the present specification, R1 to R4 are the same as or different from each other, and are each independently hydrogen, deuterium, a halogen group, a substituted or unsubstituted alkyl group having 1 to 30 carbon atoms, a substituted or unsubstituted aryl group having 6 to 60 carbon atoms, or a substituted or unsubstituted heterocyclic group having 2 to 60 carbon atoms.

In an exemplary embodiment of the present specification, R1 to R4 are the same as or different from each other, and are each independently hydrogen, deuterium, a halogen group, a substituted or unsubstituted alkyl group having 1 to 20 carbon atoms, a substituted or unsubstituted aryl group having 6 to 30 carbon atoms, or a substituted or unsubstituted heterocyclic group having 2 to 30 carbon atoms.

In an exemplary embodiment of the present specification, R1 to R4 are the same as or different from each other, and are each independently hydrogen, deuterium, a halogen group, a substituted or unsubstituted alkyl group having 1 to 10 carbon atoms, a substituted or unsubstituted aryl group having 6 to 20 carbon atoms, or a substituted or unsubstituted heterocyclic group having 2 to 20 carbon atoms.

In an exemplary embodiment of the present specification, R1 to R4 are the same as or different from each other, and are each independently hydrogen, deuterium, a halogen group, a substituted or unsubstituted alkyl group having 1 to 20 carbon atoms, a substituted or unsubstituted aryl group having 6 to 30 carbon atoms, or a substituted or unsubstituted heterocyclic group having 2 to 30 carbon atoms and containing 0, S or N.

In an exemplary embodiment of the present specification, R1 to R4 are the same as or different from each other, and are each independently hydrogen, deuterium, a halogen group, a substituted or unsubstituted alkyl group having 1 to 10 carbon atoms, a substituted or unsubstituted aryl group having 6 to 20 carbon atoms, or a substituted or unsubstituted heterocyclic group having 2 to 20 carbon atoms and containing 0, S or N.

In an exemplary embodiment of the present specification, R1 to R4 are the same as or different from each other, and are each independently hydrogen; deuterium; a halogen group; an alkyl group having 1 to 10 carbon atoms, which is unsubstituted or substituted with deuterium; an aryl group having 6 to 20 carbon atoms, which is unsubstituted or substituted with deuterium; or a heterocyclic group having 2 to 20 carbon atoms, which is unsubstituted or substituted with deuterium and containing O, S or N.

In an exemplary embodiment of the present specification, R1 to R4 are the same as or different from each other, and are each independently hydrogen, deuterium, or a substituted or unsubstituted aryl group.

In an exemplary embodiment of the present specification, R1 to R4 are the same as or different from each other, and are each independently hydrogen, deuterium, or a substituted or unsubstituted aryl group having 6 to 30 carbon atoms.

In an exemplary embodiment of the present specification, R1 to R4 are the same as or different from each other, and are each independently hydrogen, deuterium, or a substituted or unsubstituted aryl group having 6 to 20 carbon atoms.

In an exemplary embodiment of the present specification, R1 to R4 are the same as or different from each other, and are each independently hydrogen, deuterium, or a substituted or unsubstituted aryl group having 6 to 12 carbon atoms.

In an exemplary embodiment of the present specification, R1 to R4 are the same as or different from each other, and are each independently hydrogen, deuterium, or a substituted or unsubstituted aryl group having 6 to 10 carbon atoms.

In an exemplary embodiment of the present specification, R1 to R4 are the same as or different from each other, and are each independently hydrogen, deuterium, or an aryl group having 6 to 30 carbon atoms, which is unsubstituted or substituted with deuterium.

In an exemplary embodiment of the present specification, R1 to R4 are the same as or different from each other, and are each independently hydrogen, deuterium, or an aryl group having 6 to 20 carbon atoms, which is unsubstituted or substituted with deuterium.

In an exemplary embodiment of the present specification, R1 to R4 are the same as or different from each other, and are each independently hydrogen, deuterium, or an aryl group having 6 to 12 carbon atoms, which is unsubstituted or substituted with deuterium.

In an exemplary embodiment of the present specification, R1 to R4 are the same as or different from each other, and are each independently hydrogen, deuterium, or an aryl group having 6 to 10 carbon atoms, which is unsubstituted or substituted with deuterium.

In an exemplary embodiment of the present specification, R1 to R4 are the same as or different from each other, and are each independently hydrogen, deuterium, a substituted or unsubstituted phenyl group, a substituted or unsubstituted biphenyl group, a substituted or unsubstituted terphenyl group, a substituted or unsubstituted naphthyl group, or a substituted or unsubstituted fluorenyl group.

In an exemplary embodiment of the present specification, R1 to R4 are the same as or different from each other, and are each independently a substituted or unsubstituted phenyl group, a substituted or unsubstituted biphenyl group, a substituted or unsubstituted terphenyl group, a substituted or unsubstituted naphthyl group, or a substituted or unsubstituted fluorenyl group.

In an exemplary embodiment of the present specification, R1 to R4 are the same as or different from each other, and are each independently a phenyl group which is unsubstituted or substituted with deuterium; a biphenyl group which is unsubstituted or substituted with deuterium; a terphenyl group which is unsubstituted or substituted with deuterium; a naphthyl group which is unsubstituted or substituted with deuterium; or a fluorenyl group which is unsubstituted or substituted with deuterium.

In an exemplary embodiment of the present specification, R1 to R4 are the same as or different from each other, and are each independently hydrogen, deuterium, a substituted or unsubstituted phenyl group, a substituted or unsubstituted biphenyl group, or a substituted or unsubstituted naphthyl group.

In an exemplary embodiment of the present specification, R1 to R4 are the same as or different from each other, and are each independently hydrogen; deuterium; a phenyl group which is unsubstituted or substituted with deuterium; a biphenyl group which is unsubstituted or substituted with deuterium; or a naphthyl group which is unsubstituted or substituted with deuterium.

In an exemplary embodiment of the present specification, R1 to R4 are the same as or different from each other, and are each independently hydrogen, deuterium, or a substituted or unsubstituted phenyl group.

In an exemplary embodiment of the present specification, R1 to R4 are the same as or different from each other, and are each independently hydrogen, deuterium, or a phenyl group which is unsubstituted or substituted with deuterium.

In an exemplary embodiment of the present specification, R1 to R4 are the same as or different from each other, and are each independently a substituted or unsubstituted phenyl group.

In an exemplary embodiment of the present specification, R1 to R4 are the same as or different from each other, and are each independently a phenyl group which is unsubstituted or substituted with deuterium.

In an exemplary embodiment of the present specification, R1 to R4 are a phenyl group.

In an exemplary embodiment of the present specification, R′ is hydrogen, deuterium, a halogen group, a substituted or unsubstituted alkyl group, a substituted or unsubstituted aryl group, or a substituted or unsubstituted heterocyclic group.

In an exemplary embodiment of the present specification, R′ is hydrogen, deuterium, a halogen group, a substituted or unsubstituted alkyl group having 1 to 30 carbon atoms, a substituted or unsubstituted aryl group having 6 to 60 carbon atoms, or a substituted or unsubstituted heterocyclic group having 2 to 60 carbon atoms.

In an exemplary embodiment of the present specification, R′ is hydrogen, deuterium, a halogen group, a substituted or unsubstituted alkyl group having 1 to 20 carbon atoms, a substituted or unsubstituted aryl group having 6 to 30 carbon atoms, or a substituted or unsubstituted heterocyclic group having 2 to 30 carbon atoms.

In an exemplary embodiment of the present specification, R′ is hydrogen, deuterium, a halogen group, a substituted or unsubstituted alkyl group having 1 to 10 carbon atoms, a substituted or unsubstituted aryl group having 6 to 20 carbon atoms, or a substituted or unsubstituted heterocyclic group having 2 to 20 carbon atoms.

In an exemplary embodiment of the present specification, R′ is hydrogen, deuterium, a halogen group, or a substituted or unsubstituted alkyl group.

In an exemplary embodiment of the present specification, R′ is hydrogen, deuterium, a halogen group, or an alkyl group which is unsubstituted or substituted with deuterium.

In an exemplary embodiment of the present specification, R′ is hydrogen or deuterium.

In an exemplary embodiment of the present specification, R′ is hydrogen.

In an exemplary embodiment of the present specification, Np is represented by the following Chemical Formula 2:

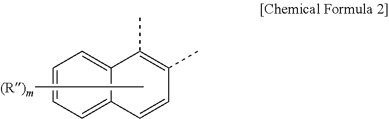

wherein in Chemical Formula 2,
the dotted lines “-----” are positions of attachment of Chemical Formula 2 where one is bonded to L1 and the other is bonded to L2 of Chemical Formula 1;
R″ is hydrogen, deuterium, a halogen group, a substituted or unsubstituted alkyl group, a substituted or unsubstituted aryl group, or a substituted or unsubstituted heterocyclic group; and
m is an integer from 0 to 6, and when m is 2 or higher, the two or more R′s are the same as or different from each other.

In an exemplary embodiment of the present specification, R″ is hydrogen, deuterium, a halogen group, a substituted or unsubstituted alkyl group having 1 to 30 carbon atoms, a substituted or unsubstituted aryl group having 6 to 60 carbon atoms, or a substituted or unsubstituted heterocyclic group having 2 to 60 carbon atoms.

In an exemplary embodiment of the present specification, R″ is hydrogen, deuterium, a halogen group, a substituted or unsubstituted alkyl group having 1 to 20 carbon atoms, a substituted or unsubstituted aryl group having 6 to 30 carbon atoms, or a substituted or unsubstituted heterocyclic group having 2 to 30 carbon atoms.

In an exemplary embodiment of the present specification, R″ is hydrogen, deuterium, a halogen group, a substituted or unsubstituted alkyl group having 1 to 10 carbon atoms, a substituted or unsubstituted aryl group having 6 to 20 carbon atoms, or a substituted or unsubstituted heterocyclic group having 2 to 20 carbon atoms.

In an exemplary embodiment of the present specification, R″ is hydrogen, deuterium, a halogen group, or a substituted or unsubstituted alkyl group.

In an exemplary embodiment of the present specification, R″ is hydrogen, deuterium, a halogen group, or an alkyl group which is unsubstituted or substituted with deuterium.

In an exemplary embodiment of the present specification, R″ is hydrogen or deuterium.

In an exemplary embodiment of the present specification, R″ is hydrogen.

In an exemplary embodiment of the present specification, Chemical Formula 1 is the following Chemical Formula 1-1 or 1-2:

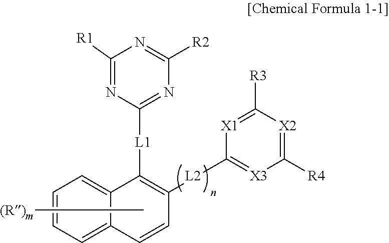

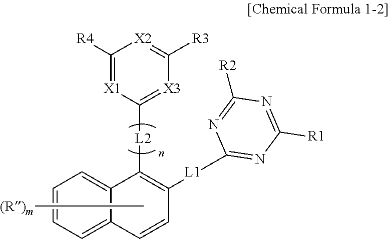

wherein in Chemical Formulae 1-1 and 1-2, the definitions of L1, L2, n, R1 to R4, X1 to X3, R″ and m are the same as the definitions in Chemical Formula 1.

In an exemplary embodiment of the present specification, Chemical Formula 1 is the following Chemical Formula 2-1 or 2-2:

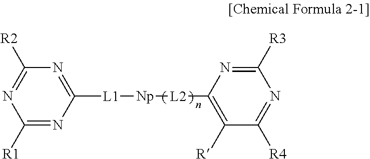

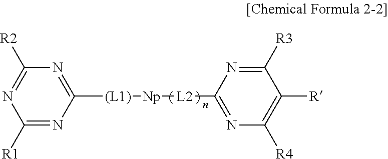

wherein in Chemical Formulae 2-1 and 2-2, the definitions of L1, L2, n, R1 to R4, R′ and Np are the same as the definitions in Chemical Formula 1.

In an exemplary embodiment of the present specification, at least 40% of the compound of Chemical Formula 1 is substituted with deuterium.

In another exemplary embodiment, 50% or more of the compound of Chemical Formula 1 is substituted with deuterium.

In still another exemplary embodiment, 60% or more of the compound of Chemical Formula 1 is substituted with deuterium.

In yet another exemplary embodiment, 70% or more of the compound of Chemical Formula 1 is substituted with deuterium.

In still yet another exemplary embodiment, 80% or more of the compound of Chemical Formula 1 is substituted with deuterium.

In a further exemplary embodiment, 90% or more of the compound of Chemical Formula 1 is substituted with deuterium.

In another further exemplary embodiment, 100% of the compound of Chemical Formula 1 is substituted with deuterium.

In an exemplary embodiment of the present specification, the compound of Chemical Formula 1 includes 40% to 60% of deuterium.

In another exemplary embodiment, the compound of Chemical Formula 1 includes 40% to 80% of deuterium.

In still another exemplary embodiment, the compound of Chemical Formula 1 includes 60% to 80% of deuterium.

In yet another exemplary embodiment, the compound of Chemical Formula 1 includes 80% to 100% of deuterium.

In an exemplary embodiment of the present specification, the compound of Chemical Formula 1 is any one of the following compounds:

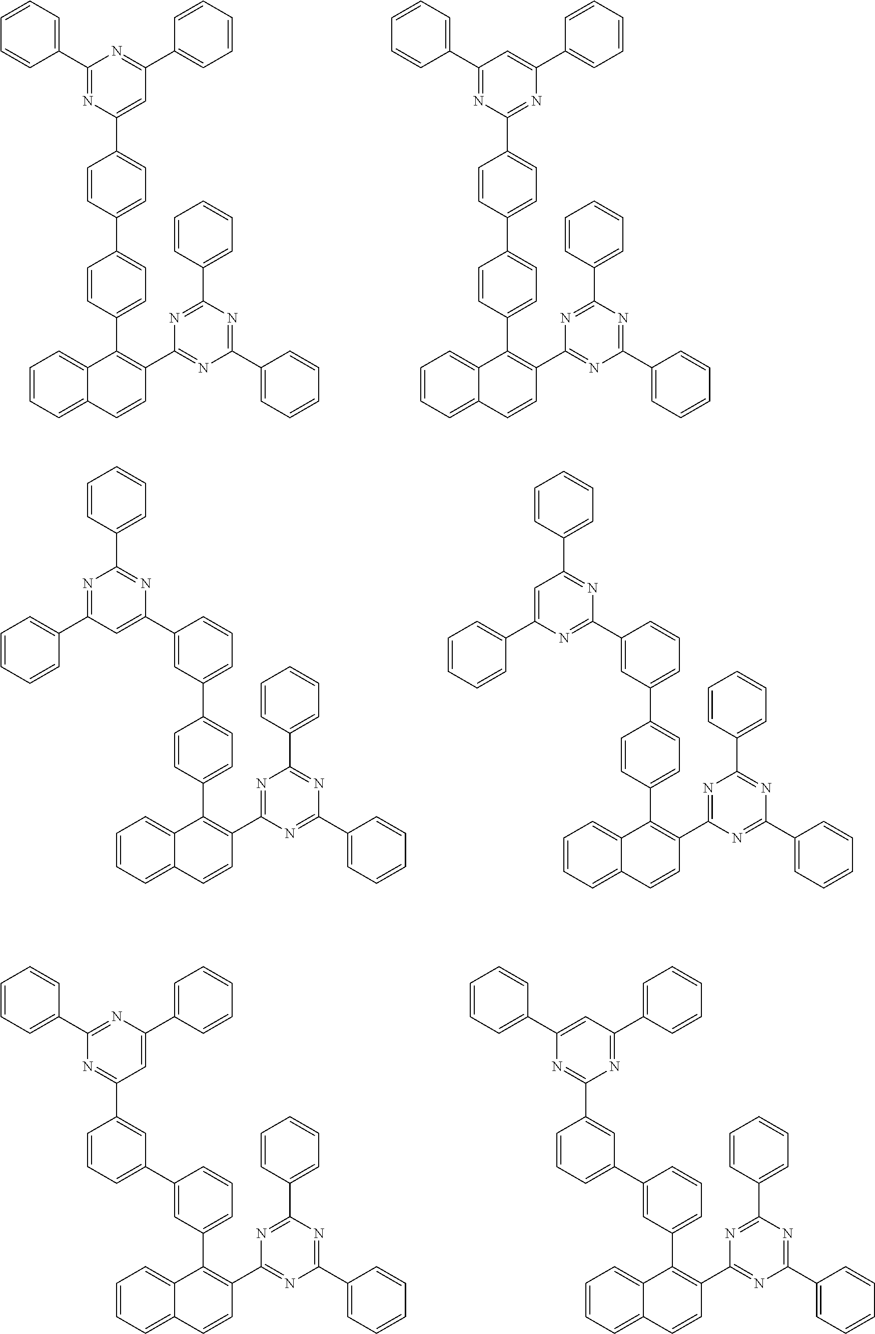

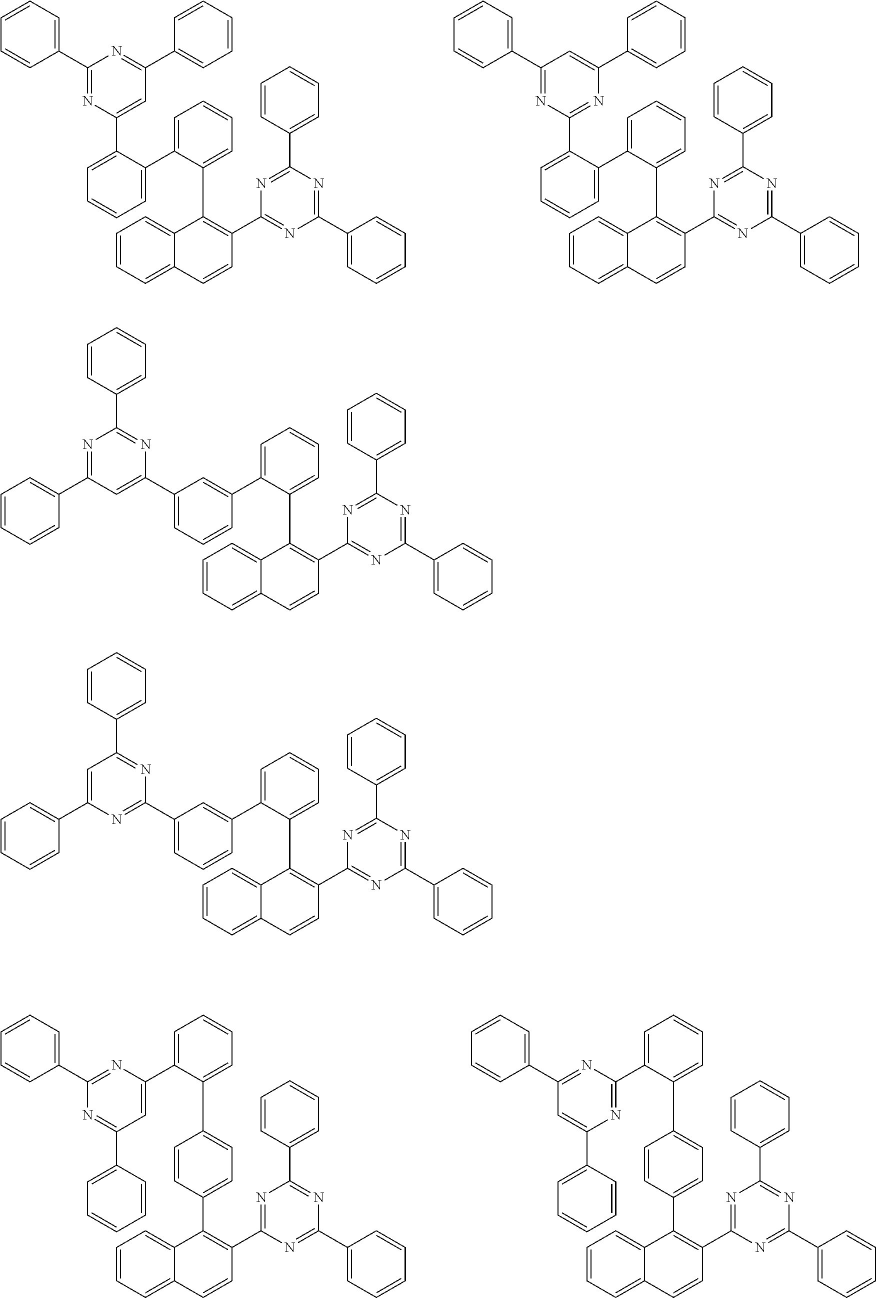

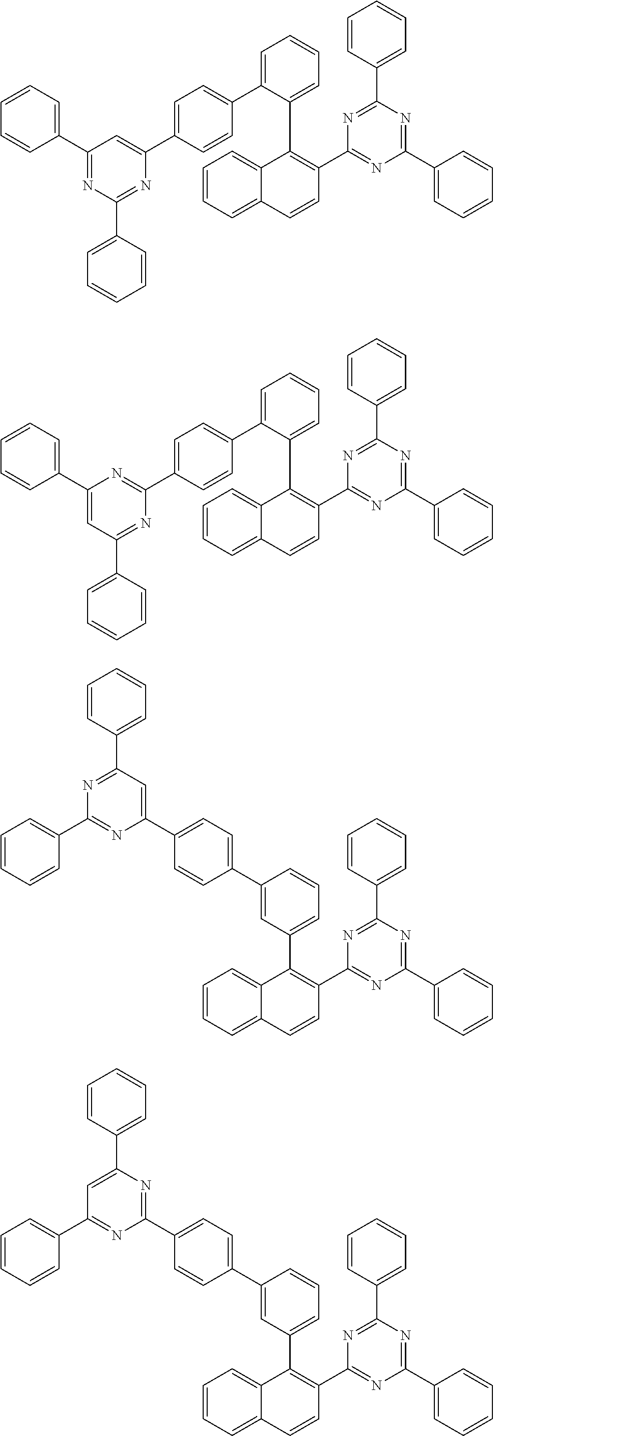

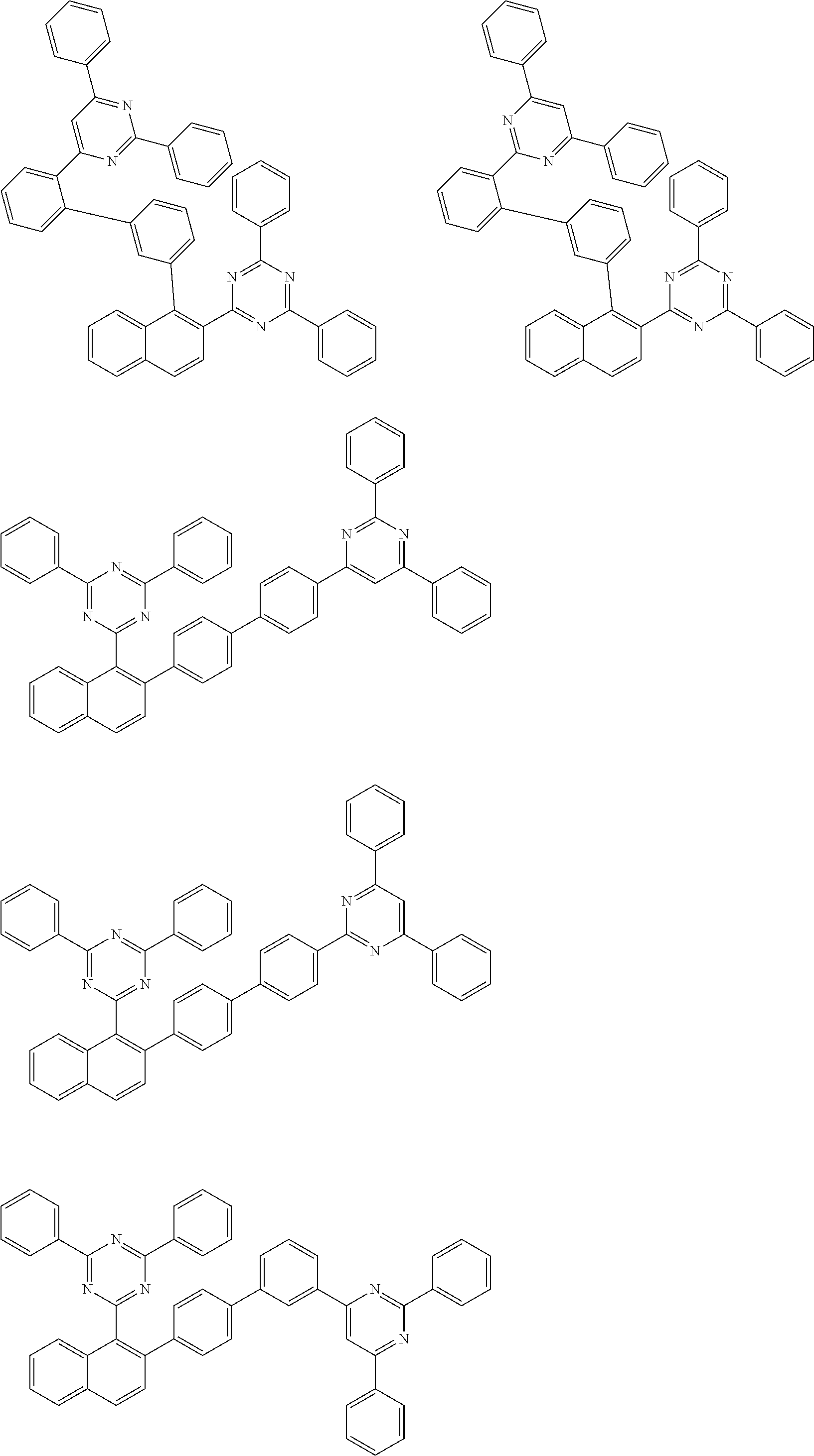

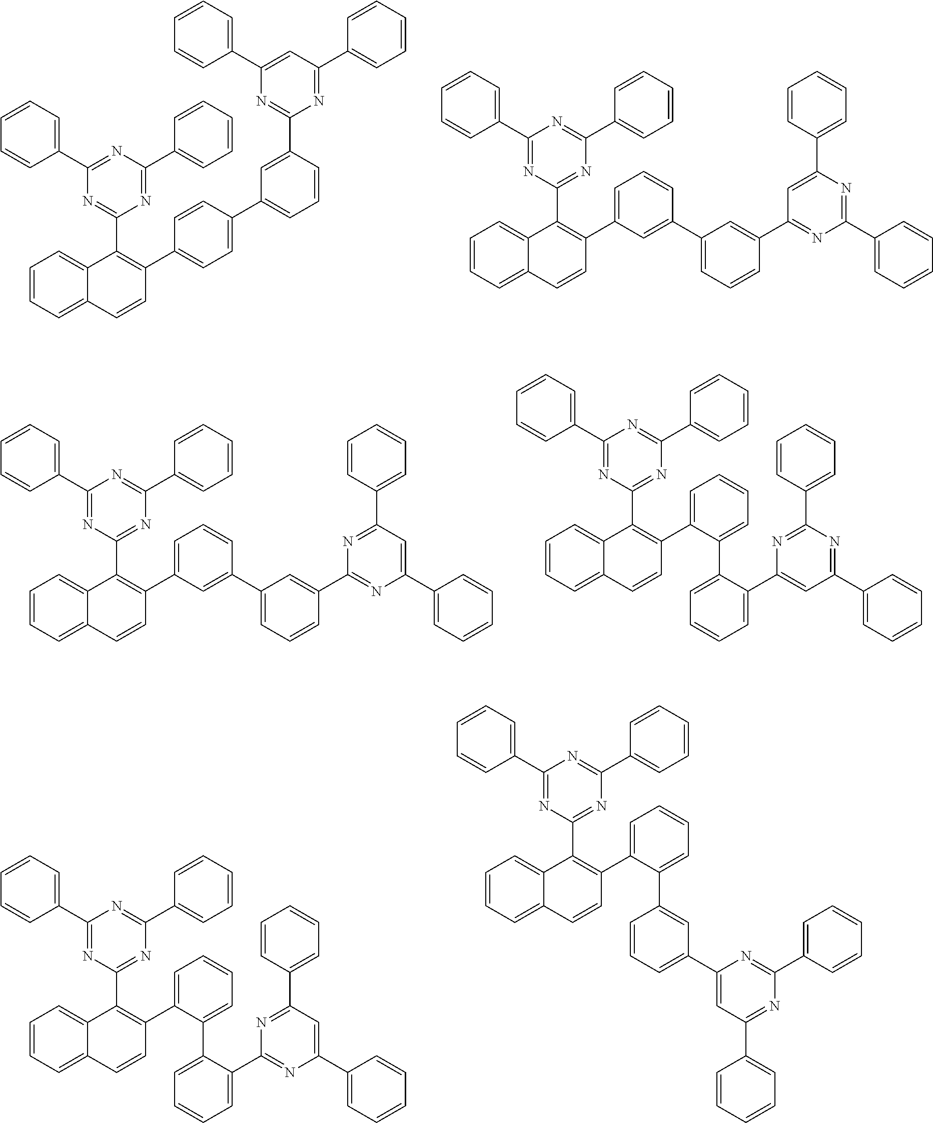

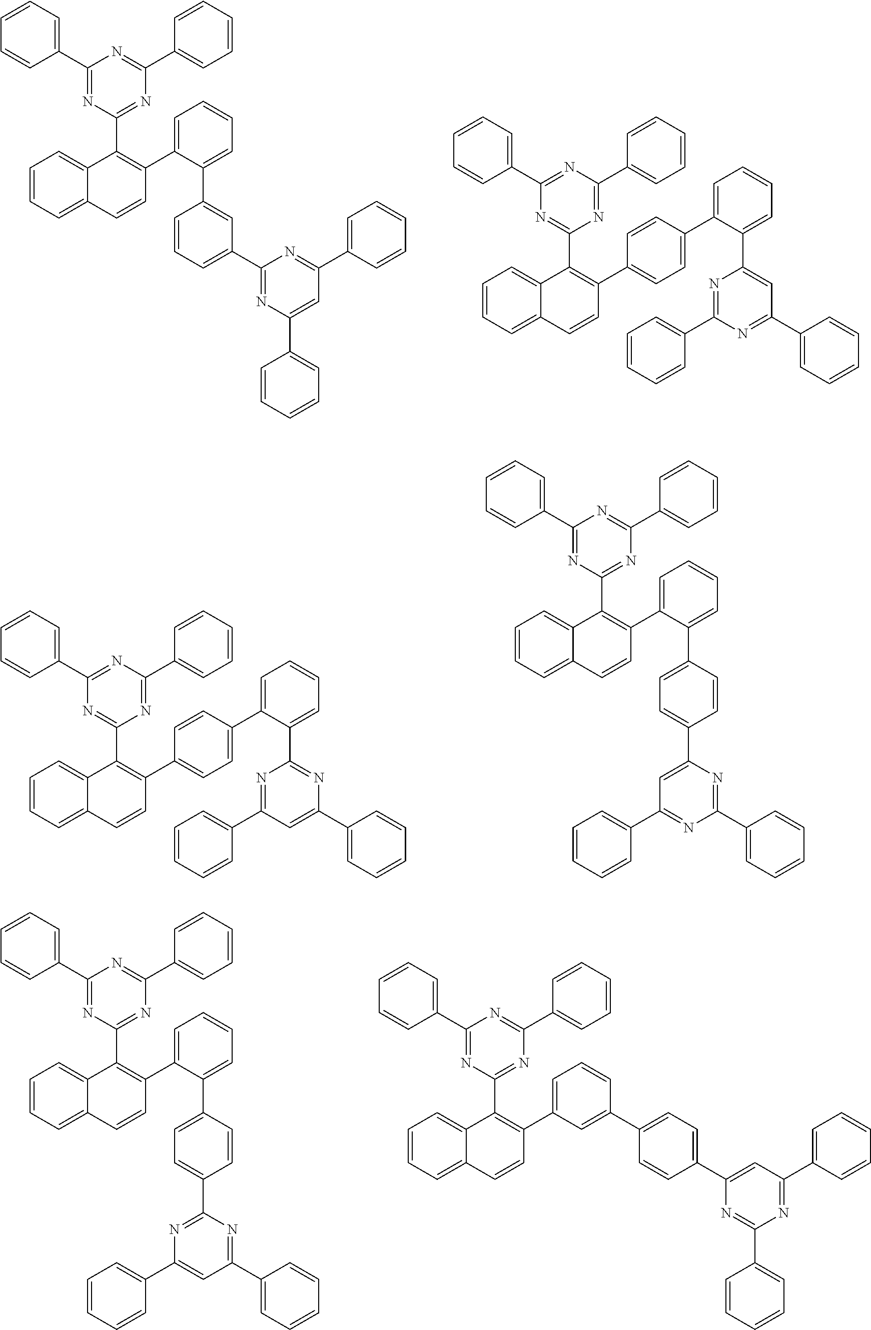

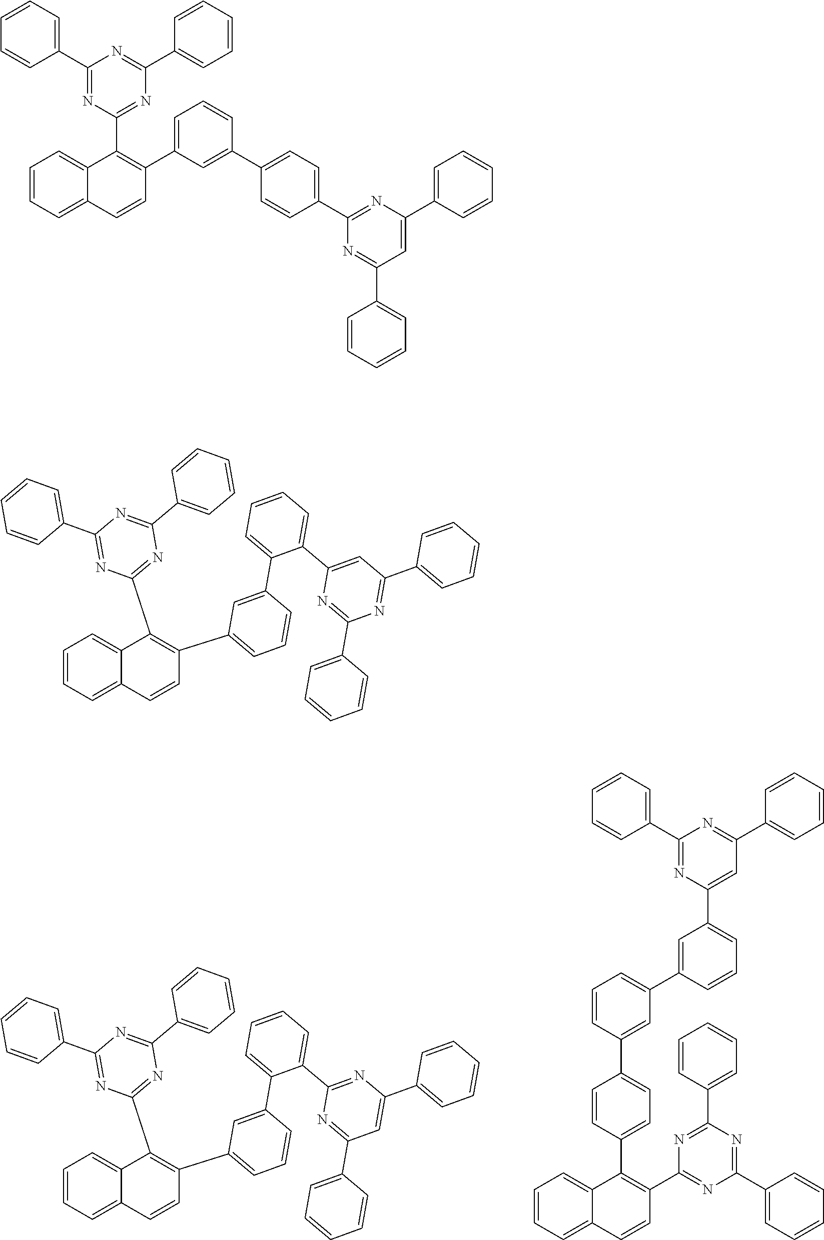 [SMILES: c1ccc(-c2cc(-c3ccccc3)nc(-c3ccc(-c4cccc(-c5ccc6ccccc6c5-c5nc(-c6ccccc6)nc(-c6ccccc6)n5)c4)cc3)n2)cc1 ; c1ccc(-c2cc(-c3ccccc3-c3cccc(-c4ccc5ccccc5c4-c4nc(-c5ccccc5)nc(-c5ccccc5)n4)c3)nc(-c3ccccc3)n2)cc1 ; c1ccc(-c2cc(-c3ccccc3)nc(-c3ccccc3-c3cccc(-c4ccc5ccccc5c4-c4nc(-c5ccccc5)nc(-c5ccccc5)n4)c3)n2)cc1 ; c1ccc(-c2cc(-c3cccc(-c4cccc(-c5ccc(-c6c(-c7nc(-c8ccccc8)nc(-c8ccccc8)n7)ccc7ccccc67)cc5)c4)c3)nc(-c3ccccc3)n2)cc1]

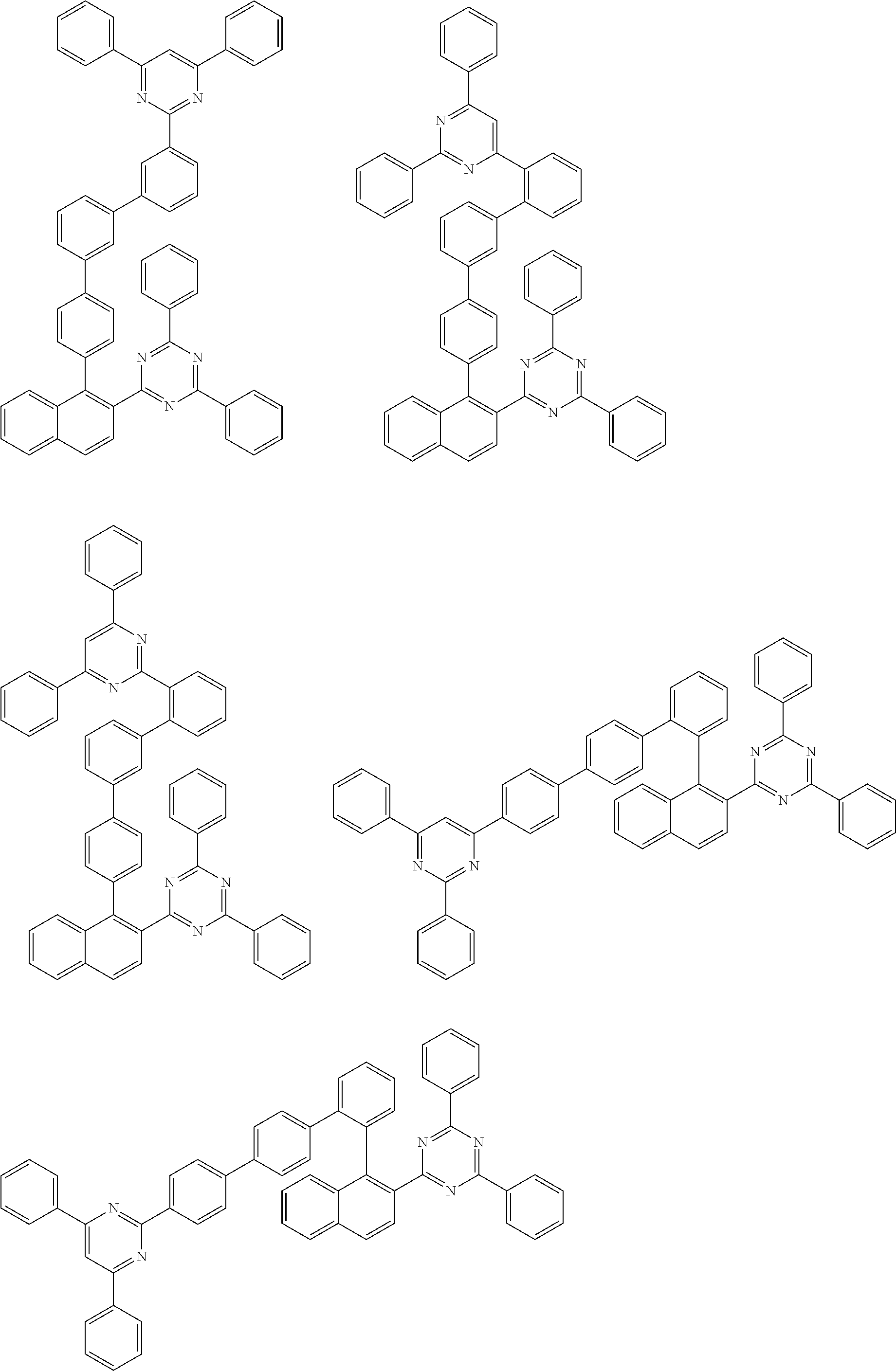

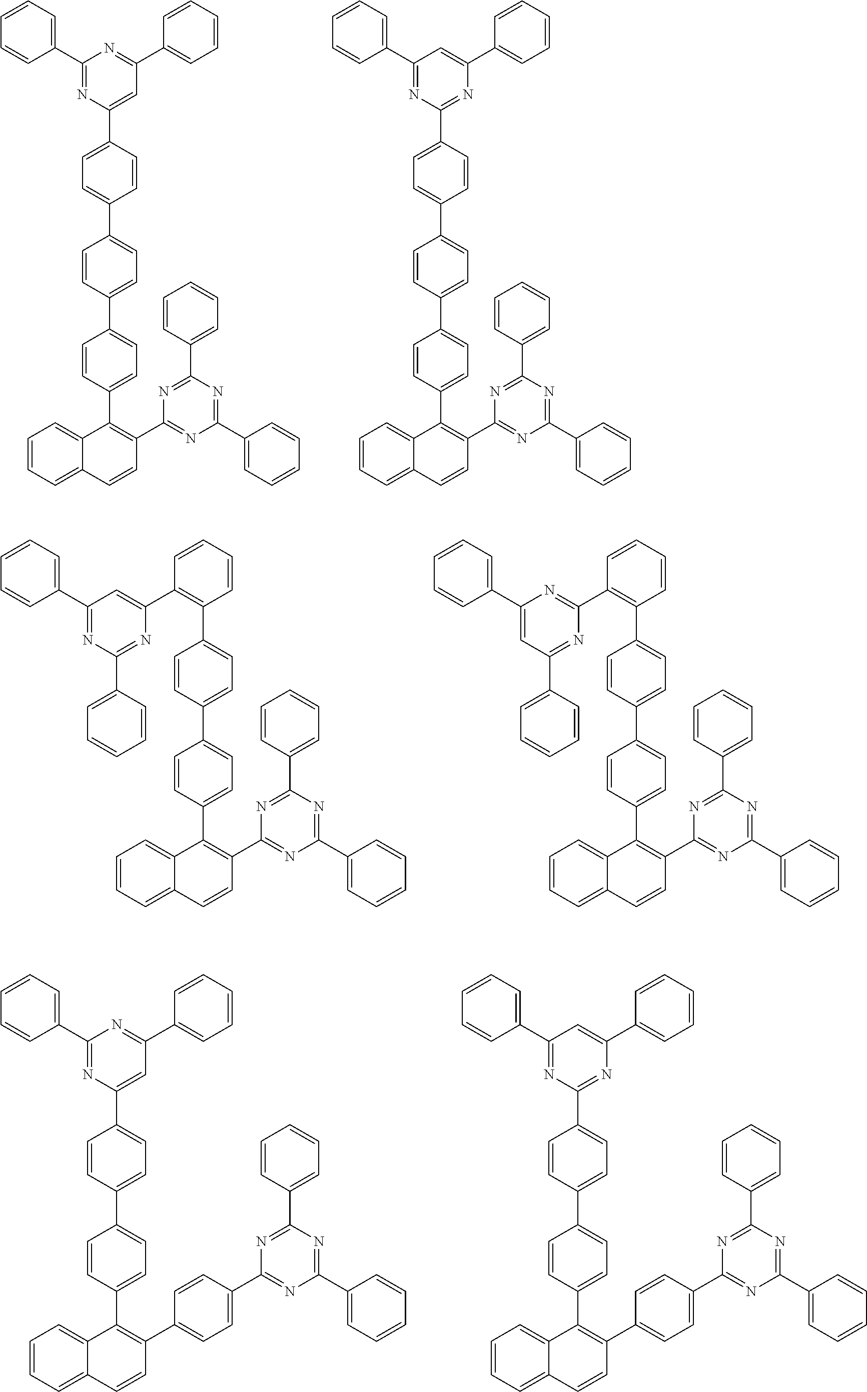

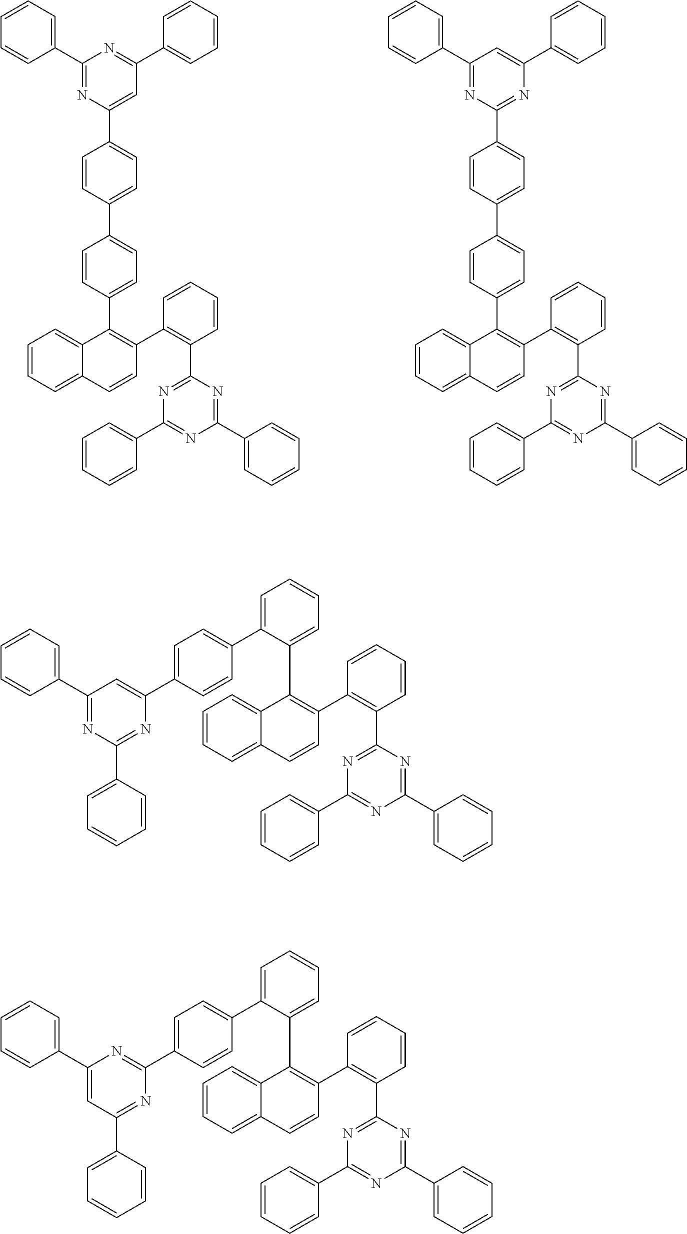 [SMILES: c1ccc(-c2cc(-c3ccc(-c4ccc(-c5c(-c6ccccc6-c6nc(-c7ccccc7)nc(-c7ccccc7)n6)ccc6ccccc56)cc4)cc3)nc(-c3ccccc3)n2)cc1 ; c1ccc(-c2cc(-c3ccccc3)nc(-c3ccc(-c4ccc(-c5c(-c6ccccc6-c6nc(-c7ccccc7)nc(-c7ccccc7)n6)ccc6ccccc56)cc4)cc3)n2)cc1 ; c1ccc(-c2cc(-c3ccc(-c4ccccc4-c4c(-c5ccccc5-c5nc(-c6ccccc6)nc(-c6ccccc6)n5)ccc5ccccc45)cc3)nc(-c3ccccc3)n2)cc1 ; c1ccc(-c2cc(-c3ccccc3)nc(-c3ccc(-c4ccccc4-c4c(-c5ccccc5-c5nc(-c6ccccc6)nc(-c6ccccc6)n5)ccc5ccccc45)cc3)n2)cc1]

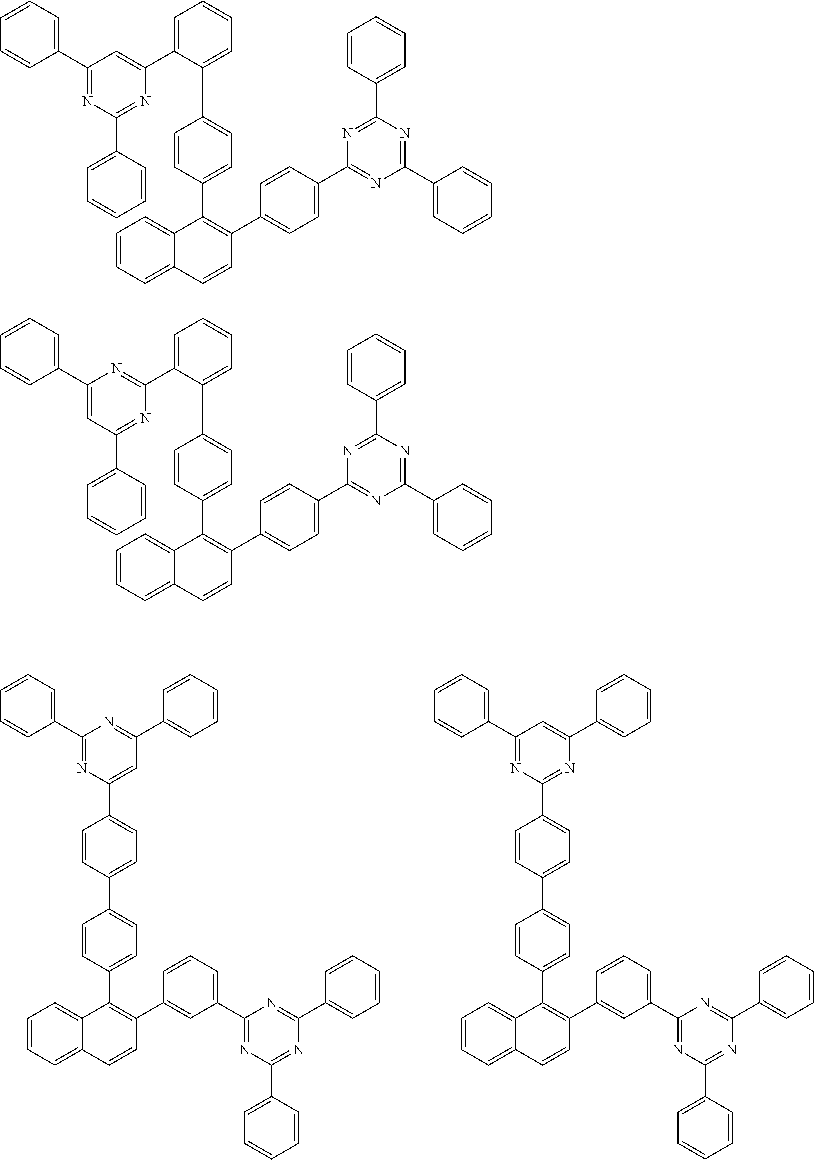

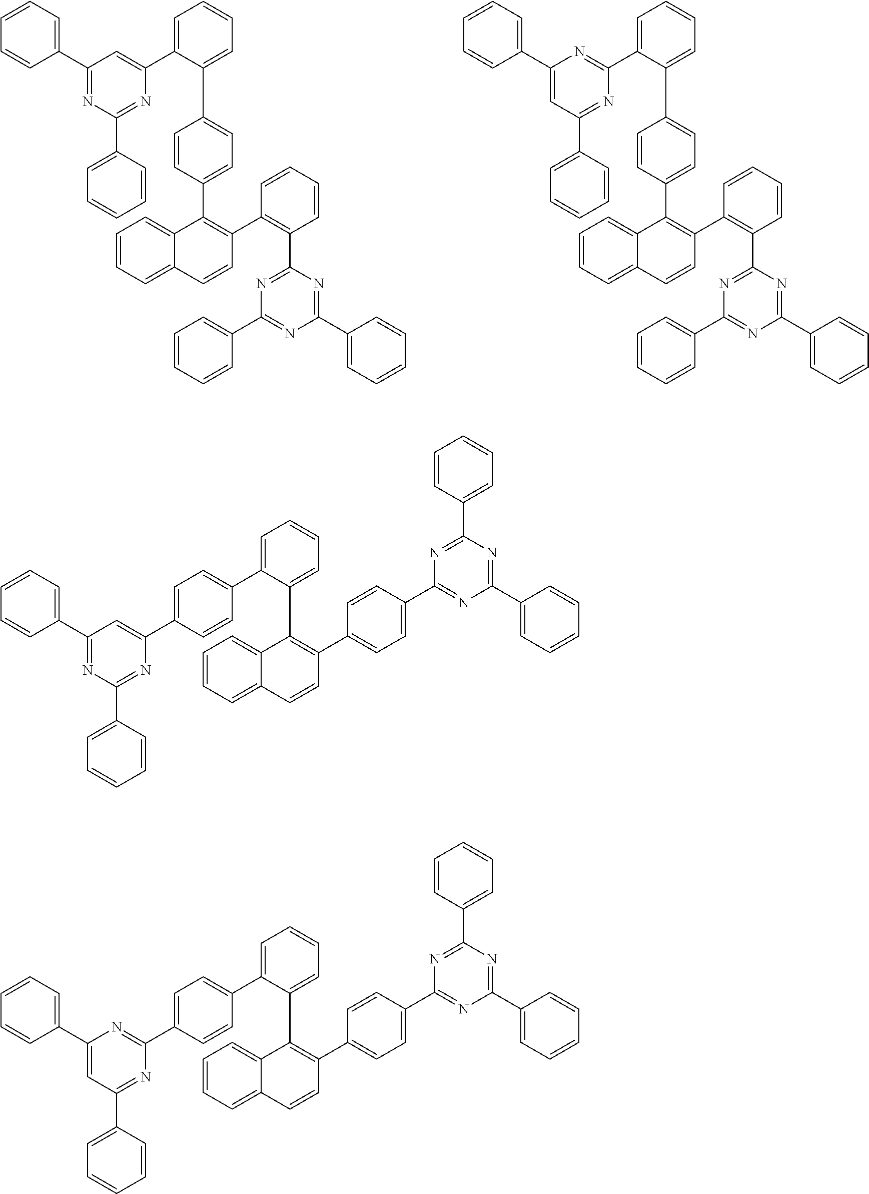 [SMILES: c1ccc(-c2cc(-c3ccccc3-c3ccc(-c4c(-c5ccccc5-c5nc(-c6ccccc6)nc(-c6ccccc6)n5)ccc5ccccc45)cc3)nc(-c3ccccc3)n2)cc1 ; c1ccc(-c2cc(-c3ccccc3)nc(-c3ccccc3-c3ccc(-c4c(-c5ccccc5-c5nc(-c6ccccc6)nc(-c6ccccc6)n5)ccc5ccccc45)cc3)n2)cc1 ; c1ccc(-c2cc(-c3ccc(-c4ccccc4-c4c(-c5ccc(-c6nc(-c7ccccc7)nc(-c7ccccc7)n6)cc5)ccc5ccccc45)cc3)nc(-c3ccccc3)n2)cc1 ; c1ccc(-c2cc(-c3ccccc3)nc(-c3ccc(-c4ccccc4-c4c(-c5ccc(-c6nc(-c7ccccc7)nc(-c7ccccc7)n6)cc5)ccc5ccccc45)cc3)n2)cc1]

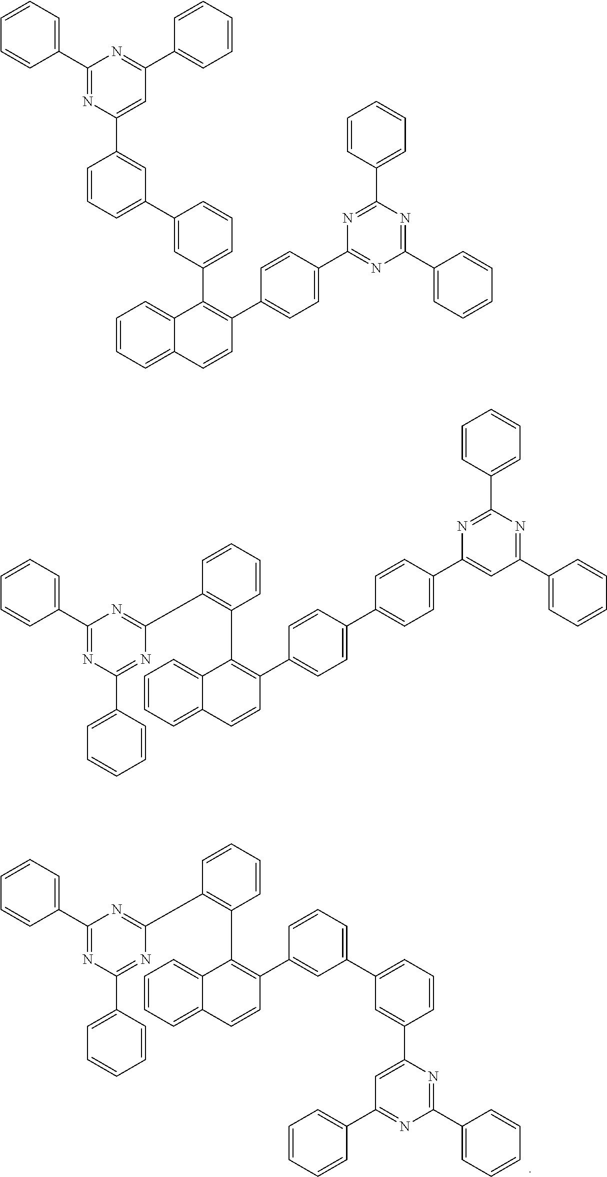 [SMILES: c1ccc(-c2cc(-c3cccc(-c4cccc(-c5c(-c6ccc(-c7nc(-c8ccccc8)nc(-c8ccccc8)n7)cc6)ccc6ccccc56)c4)c3)nc(-c3ccccc3)n2)cc1 ; c1ccc(-c2cc(-c3ccc(-c4ccc(-c5ccc6ccccc6c5-c5ccccc5-c5nc(-c6ccccc6)nc(-c6ccccc6)n5)cc4)cc3)nc(-c3ccccc3)n2)cc1 ; c1ccc(-c2cc(-c3cccc(-c4cccc(-c5ccc6ccccc6c5-c5ccccc5-c5nc(-c6ccccc6)nc(-c6ccccc6)n5)c4)c3)nc(-c3ccccc3)n2)cc1]

A core structure can be prepared as in the method of the Preparation Example to be described below from the compound of Chemical Formula 1 according to an exemplary embodiment of the present specification. The substituent can be bonded by a method known in the art, and the kind and position of the substituent or the number of substituents can be changed according to the technology known in the art.

In the present specification, compounds having various energy band gaps can be synthesized by introducing various substituents into the core structure of the compound of Chemical Formula 1. Further, in the present specification, various substituents can be introduced into the core structures having the structure described above to adjust the HOMO and LUMO energy levels of a compound.

Further, the present specification provides an organic light emitting device including the above-described compound.

The organic light emitting device according to the present specification is an organic light emitting device including: an anode; a cathode; and an organic material layer having one or more layers provided between the anode and the cathode, in which one or more layers of the organic material layer include the above-described compound of Chemical Formula 1.

The organic light emitting device of the present specification can be manufactured using typical manufacturing methods and materials of an organic light emitting device, except that the above-described compound of Chemical Formula 1 is used to form an organic material layer.

The compound can be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

The organic material layer of the organic light emitting device of the present specification can be composed of a single-layered structure, but can also be composed of a multi-layered structure in which two or more organic material layers are stacked. For example, the organic light emitting device of the present invention can have a structure including one or more layers of a hole transport layer, a hole injection layer, an electron blocking layer, a hole injection and transport layer, an electron transport layer, an electron injection layer, a hole blocking layer, and an electron injection and transport layer as organic material layers. However, the structure of the organic light emitting device of the present specification is not limited thereto, and can include a fewer or greater number of organic material layers.

In an exemplary embodiment of the present application, the organic material layer includes a light emitting layer, and the light emitting layer includes the compound.

In an exemplary embodiment of the present application, the organic material layer includes a hole injection layer or a hole transport layer, and the hole injection layer or the hole transport layer includes the compound.

In another exemplary embodiment, the organic material layer includes a light emitting layer, and the light emitting layer includes the compound.

In an exemplary embodiment of the present application, the organic material layer includes an electron transport layer or an electron injection layer, and the electron transport layer or the electron injection layer includes the compound.

In an exemplary embodiment of the present application, the organic material layer includes an electron transport layer, and the electron transport layer includes the compound.

In an exemplary embodiment of the present application, the organic material layer includes an electron blocking layer or a hole blocking layer, and the electron blocking layer or the hole blocking layer includes the compound.

In an exemplary embodiment of the present application, the organic material layer is an electron transport layer, and the organic light emitting device further includes one or two or more layers selected from the group consisting of a hole injection layer, a hole transport layer, a light emitting layer, an electron injection layer, an electron blocking layer, and a hole blocking layer.

In an exemplary embodiment of the present application, the organic light emitting device includes: a first electrode; a second electrode provided to face the first electrode; a light emitting layer provided between the first electrode and the second electrode; and an organic material layer having two or more layers provided between the light emitting layer and the first electrode, or between the light emitting layer and the second electrode, in which at least one of the organic material layers having two or more layers includes the compound. In an exemplary embodiment of the present application, as the organic material layer having two or more layers, two or more can be selected from the group consisting of an electron transport layer, an electron injection layer, a layer which transports and injects electrons simultaneously, and a hole blocking layer.

In an exemplary embodiment of the present application, the organic material layer includes two or more electron transport layers, and at least one of the two or more electron transport layers includes the compound. Specifically, in an exemplary embodiment of the present specification, the compound can also be included in one layer of the electron transport layer having two or more layers, and can be included in each of the electron transport layer having two or more layers. Further, in an exemplary embodiment of the present application, when the compound is included in each of the electron transport layer having two or more layers, materials other than the compound can be the same as or different from each other.

In an exemplary embodiment of the present application, the organic material layer includes an electron injection and transport layer, and the electron injection and transport layer includes the compound.

In an exemplary embodiment of the present application, the organic material layer further includes a hole injection layer or a hole transport layer, which includes a compound including an arylamino group, a carbazolyl group, or a benzocarbazolyl group, in addition to the organic material layer including the compound.

In another exemplary embodiment, the organic light emitting device can be a normal type organic light emitting device in which a positive electrode, an organic material layer having one or more layers, and a negative electrode are sequentially stacked on a substrate.

In still another exemplary embodiment, the organic light emitting device can be an inverted type organic light emitting device in which a negative electrode, an organic material layer having one or more layers, and a positive electrode are sequentially stacked on a substrate.

The organic light emitting device can have, for example, the stacking structure described below, but the stacking structure is not limited thereto.
(1) Anode/Hole transport layer/Light emitting layer/Cathode
(2) Anode/Hole injection layer/Hole transport layer/Light emitting layer/Cathode
(3) Anode/Hole injection layer/Hole buffer layer/Hole transport layer/Light emitting layer/Cathode
(4) Anode/Hole transport layer/Light emitting layer/Electron transport layer/Cathode
(5) Anode/Hole transport layer/Light emitting layer/Electron transport layer/Electron injection layer/Cathode
(6) Anode/Hole injection layer/Hole transport layer/Light emitting layer/Electron transport layer/Cathode
(7) Anode/Hole injection layer/Hole transport layer/Light emitting layer/Electron transport layer/Electron injection layer/Cathode
(8) Anode/Hole injection layer/Hole buffer layer/Hole transport layer/Light emitting layer/Electron transport layer/Cathode
(9) Anode/Hole injection layer/Hole buffer layer/Hole transport layer/Light emitting layer/Electron transport layer/Electron injection layer/Cathode
(10) Anode/Hole transport layer/Electron blocking layer/Light emitting layer/Electron transport layer/Cathode
(11) Anode/Hole transport layer/Electron blocking layer/Light emitting layer/Electron transport layer/Electron injection layer/Cathode
(12) Anode/Hole injection layer/Hole transport layer/Electron blocking layer/Light emitting layer/Electron transport layer/Cathode
(13) Anode/Hole injection layer/Hole transport layer/Electron blocking layer/Light emitting layer/Electron transport layer/Electron injection layer/Cathode
(14) Anode/Hole transport layer/Light emitting layer/Hole blocking layer/Electron transport layer/Cathode
(15) Anode/Hole transport layer/Light emitting layer/Hole blocking layer/Electron transport layer/Electron injection layer/Cathode
(16) Anode/Hole injection layer/Hole transport layer/Light emitting layer/Hole blocking layer/Electron transport layer/Cathode
(17) Anode/Hole injection layer/Hole transport layer/Light emitting layer/Hole blocking layer/Electron transport layer/Electron injection layer/Cathode
(18) Anode/Hole injection layer/Hole transport layer/Electron blocking layer/Light emitting layer/Hole blocking layer/Electron transport layer/Electron injection layer/Cathode/Capping layer

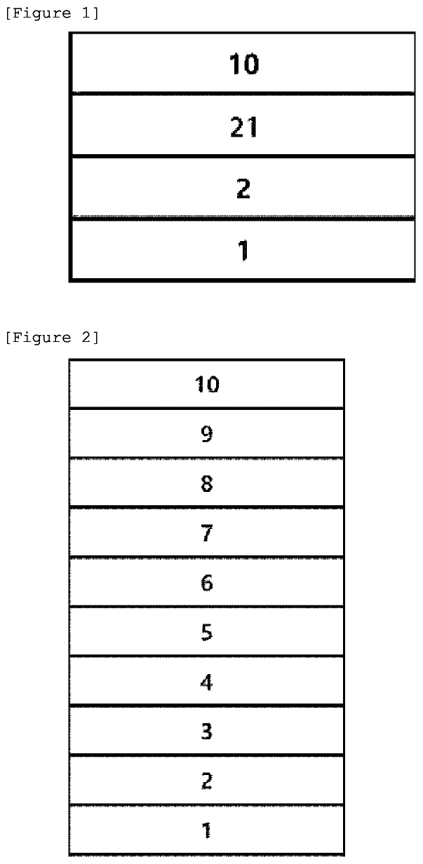

The structure of the organic light emitting device of the present specification can have structures illustrated in FIGS. 1 to 3, but is not limited thereto.

FIG. 1 illustrates an example of an organic light emitting device in which a substrate 1, an anode 2, an organic material layer 21, and a cathode 10 are sequentially stacked. In the structure described above, the compound can be included in the organic material layer 21.

FIG. 2 illustrates an example of an organic light emitting device in which a substrate 1, an anode 2, a hole injection layer 3, a hole transport layer 4, an electron blocking layer 5, a light emitting layer 6, a hole blocking layer 7, an electron transport layer 8, an electron injection layer 9, and a cathode 10 are sequentially stacked. In the structure described above, the compound can be included in the hole injection layer 3, the hole transport layer 4, the electron blocking layer 5, the light emitting layer 6, the hole blocking layer 7, the electron transport layer 8, or the electron injection layer 9.

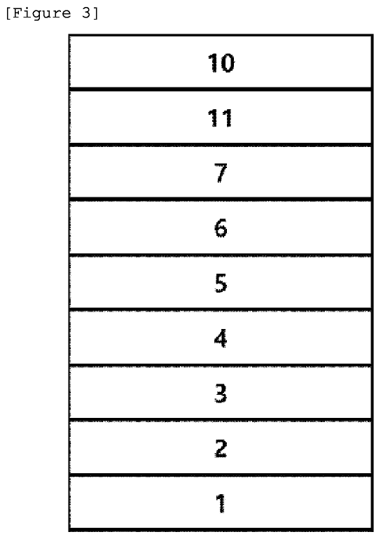

FIG. 3 illustrates an example of an organic light emitting device in which a substrate 1, an anode 2, a hole injection layer 3, a hole transport layer 4, an electron blocking layer 5, a light emitting layer 6, a hole blocking layer 7, an electron injection and transport layer 11, and a cathode 10 are sequentially stacked. In the structure described above, the compound can be included in the hole injection layer 3, the hole transport layer 4, the electron blocking layer 5, the light emitting layer 6, the hole blocking layer 7, or the electron injection and transport layer 11.

In the structure described above, the compound can be included in one or more layers of the hole injection layer, the hole transport layer, the light emitting layer, and the electron transport layer.

In the structure described above, the compound can be included in the electron injection and transport layer.

The organic light emitting device of the present application can be manufactured by the materials and methods known in the art, except that one or more layers of the organic material layer include the compound of the present application, that is, the compound.

When the organic light emitting device includes a plurality of organic material layers, the organic material layers can be formed of the same material or different materials.

The organic light emitting device of the present application can be manufactured by the materials and methods known in the art, except that one or more layers of the organic material layer include the compound, that is, the compound of Chemical Formula 1.

For example, the organic light emitting device of the present application can be manufactured by sequentially stacking a first electrode, an organic material layer, and a second electrode on a substrate. In this case, the organic light emitting device of the present application can be manufactured by depositing a metal or a metal oxide having conductivity, or an alloy thereof on a substrate to form a positive electrode, forming an organic material layer including a hole injection layer, a hole transport layer, a light emitting layer, and an electron transport layer thereon, and then depositing a material, which can be used as a negative electrode, thereon, by using a physical vapor deposition (PVD) method such as sputtering or e-beam evaporation. In addition to the method described above, an organic light emitting device can be made by sequentially depositing a negative electrode material, an organic material layer, and a positive electrode material on a substrate.

Further, the compound of Chemical Formula 1 can be formed as an organic material layer by not only a vacuum deposition method, but also a solution application method when an organic light emitting device is manufactured. Here, the solution application method means spin coating, dip coating, doctor blading, inkjet printing, screen printing, a spray method, roll coating, and the like, but is not limited thereto.

In addition to the method described above, an organic light emitting device can also be made by sequentially depositing a negative electrode material, an organic material layer, and a positive electrode material on a substrate (International Publication No. 2003/012890). However, the manufacturing method is not limited thereto.

As the positive electrode material, materials having a high work function are usually preferred so as to facilitate the injection of holes into an organic material layer. Specific examples of the positive electrode material which can be used in the present invention include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SNO₂:Sb; a conductive polymer such as poly(3-methylthiophene), poly[3,4-(ethylene-1,2-dioxy)thiophene] (PEDOT), polypyrrole, and polyaniline; and the like, but are not limited thereto.

As the negative electrode material, materials having a low work function are usually preferred so as to facilitate the injection of electrons into an organic material layer. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

The hole injection layer is a layer which injects holes from an electrode, and a hole injection material is preferably a compound which has a capability of transporting holes and thus has an effect of injecting holes at a positive electrode and an excellent effect of injecting holes for a light emitting layer or a light emitting material, prevents excitons produced from the light emitting layer from moving to an electron injection layer or an electron injection material, and is also excellent in the ability to form a thin film. The highest occupied molecular orbital (HOMO) of the hole injection material is preferably a value between the work function of the positive electrode material and the HOMO of the neighboring organic material layer. Specific examples of the hole injection material include metal porphyrin, oligothiophene, arylamine-based organic materials, hexanitrile hexaazatriphenylene-based organic materials, quinacridone-based organic materials, perylene-based organic materials, anthraquinone, polyaniline-based and polythiophene-based electrically conductive polymers, and the like, but are not limited thereto.

The hole transport layer is a layer which accepts holes from a hole injection layer and transports the holes to a light emitting layer, and a hole transport material is suitably a material having high hole mobility which can accept holes from a positive electrode or a hole injection layer and transfer the holes to a light emitting layer. Specific examples thereof include arylamine-based organic materials, conductive polymers, block copolymers having both conjugated portions and non-conjugated portions, and the like, but are not limited thereto.

The light emitting material is a material which can receive holes and electrons from a hole transport layer and an electron transport layer, and combine the holes and the electrons to emit light in a visible ray region, and is preferably a material having high quantum efficiency for fluorescence or phosphorescence. Specific examples thereof include: 8-hydroxy-quinoline aluminum complexes (Alq₃); carbazole-based compounds; dimerized styryl compounds; BAlq; 10-hydroxybenzoquinoline-metal compounds; benzoxazole-based, benzothiazole-based and benzimidazole-based compounds; poly(p-phenylenevinylene) (PPV)-based polymers; spiro compounds; polyfluorene, rubrene, and the like, but are not limited thereto.

The light emitting layer can include a host material and a dopant material. Examples of the host material include fused aromatic ring derivatives, or hetero ring-containing compounds, and the like. Specific examples of the fused aromatic ring derivative include an anthracene derivative, a pyrene derivative, a naphthalene derivative, a pentacene derivative, a phenanthrene compound, a fluoranthene compound, and the like, and specific examples of the hetero ring-containing compound include a compound, a dibenzofuran derivative, a ladder-type furan compound, a pyrimidine derivative, and the like, but the examples are not limited thereto.

The electron transport material is a material which receives electrons from an electron injection layer and transports the electrons to a light emitting layer, and an electron transport material is suitably a material which can inject electrons well from a negative electrode and can transfer the electrons to a light emitting layer, and has large mobility for the electrons. Specific examples thereof include: Al complexes of 8-hydroxyquinoline; complexes including Alq₃; organic radical compounds; hydroxyflavone-metal complexes, and the like, but are not limited thereto. The electron transport layer can be used with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

In an exemplary embodiment of the present specification, the electron transport layer can include a compound of Chemical Formula 1 of the present invention, and can further include an n-type dopant or organic metal compound. According to an example, the n-type dopant or organic metal compound can be LiQ, and the compound of Chemical Formula 1 of the present invention and the n-type dopant (or organic metal compound) can be included at a weight ratio of 2:8 to 8:2, for example, 4:6 to 6:4.

The electron injection layer is a layer which injects electrons from an electrode, and an electron injection material is preferably a compound which has a capability of transporting electrons, an effect of injecting electrons from a negative electrode, and an excellent effect of injecting electrons into a light emitting layer or a light emitting material, prevents excitons produced from a light emitting layer from moving to a hole injection layer, and is also excellent in the ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, metal complex compounds, nitrogen-containing 5-membered ring derivatives, and the like, but are not limited thereto.

In an exemplary embodiment of the present specification, as an electron injection and transport layer material, the electron transport material and/or the electron injection material can be used.

Examples of the metal complex compounds include 8-hydroxyquinolinato lithium, bis(8-hydroxyquinolinato) zinc, bis(8-hydroxyquinolinato) copper, bis(8-hydroxyquinolinato) manganese, tris(8-hydroxyquinolinato) aluminum, tris(2-methyl-8-hydroxyquinolinato) aluminum, tris(8-hydroxyquinolinato) gallium, bis(10-hydroxybenzo[h]quinolinato) beryllium, bis(10-hydroxybenzo[h]quinolinato) zinc, bis(2-methyl-8-quinolinato) chlorogallium, bis(2-methyl-8-quinolinato) (o-cresolato) gallium, bis(2-methyl-8-quinolinato) (1-naphtholato) aluminum, bis(2-methyl-8-quinolinato) (2-naphtholato) gallium, and the like, but are not limited thereto.

The hole blocking layer is a layer which blocks holes from reaching a negative electrode, and can be generally formed under the same conditions as those of the hole injection layer. Specific examples thereof include oxadiazole derivatives or triazole derivatives, phenanthroline derivatives, BCP, aluminum complexes, and the like, but are not limited thereto.

The organic light emitting device according to the present specification can be a top emission type, a bottom emission type, or a dual emission type according to the materials to be used.

### EXAMPLES

Hereinafter, the present specification will be described in detail with reference to Examples for specifically describing the present specification. However, the Examples according to the present specification can be modified in various forms, and it is not interpreted that the scope of the present application is limited to the Examples described in detail below. The Examples of the present application are provided to explain the present specification more completely to a person with ordinary skill in the art.

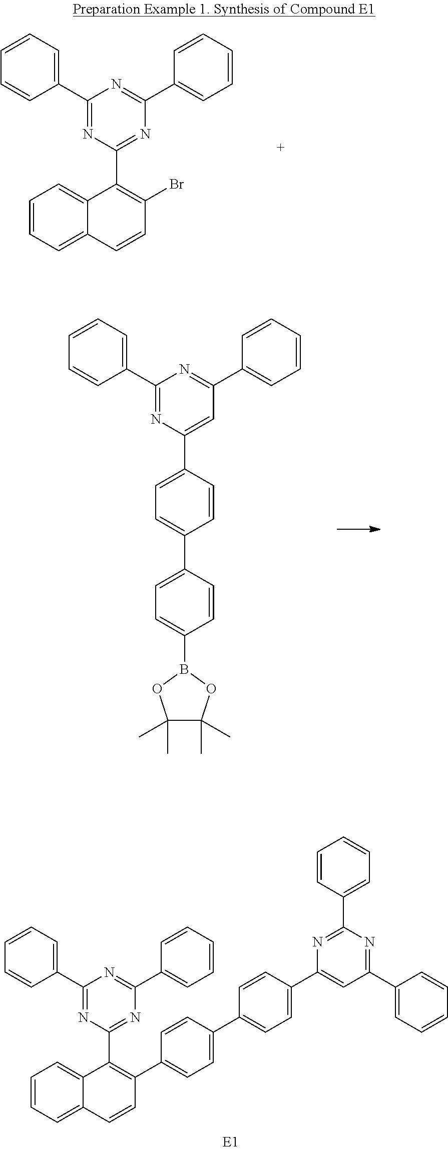 [SMILES: Brc1ccc2ccccc2c1-c1nc(-c2ccccc2)nc(-c2ccccc2)n1 ; CC1(C)OB(c2ccc(-c3ccc(-c4cc(-c5ccccc5)nc(-c5ccccc5)n4)cc3)cc2)OC1(C)C ; c1ccc(-c2cc(-c3ccc(-c4ccc(-c5ccc6ccccc6c5-c5nc(-c6ccccc6)nc(-c6ccccc6)n5)cc4)cc3)nc(-c3ccccc3)n2)cc1]

After the compound 2-(2-bromonaphthalen-1-yl)-4,6-diphenyl-1,3,5-triazine (10.0 g, 22.8 mmol) and 2,4-diphenyl-6-(4′-(4,4,5,5-tetramethyl-1,3,2-dioxaborolan-2-yl)-[1,1′-biphenyl]-4-yl)pyrimidine (11.64 g, 22.8 mmol) were completely dissolved in tetrahydrofuran (100 ml), potassium carbonate (15.7 g, 114 mmol) dissolved in 75 ml of water was added thereto, and tetrakistriphenylphosphinepalladium (476 mg, 0.8 mmol) dissolved in tetrahydrofuran was slowly introduced thereinto. The temperature was lowered to room temperature, the reaction was terminated, and then the potassium carbonate solution was removed by filtering the white solid. The filtered white solid was washed each twice with water and ethyl acetate to prepare Compound E1 (15.2 g, yield 90%).

MS[M+H]⁺=741

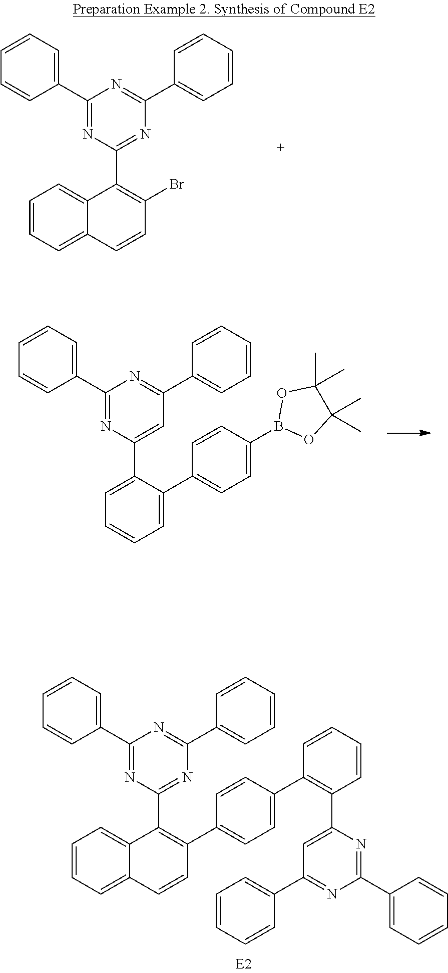 [SMILES: Brc1ccc2ccccc2c1-c1nc(-c2ccccc2)nc(-c2ccccc2)n1 ; CC1(C)OB(c2ccc(-c3ccccc3-c3cc(-c4ccccc4)nc(-c4ccccc4)n3)cc2)OC1(C)C ; c1ccc(-c2cc(-c3ccccc3-c3ccc(-c4ccc5ccccc5c4-c4nc(-c5ccccc5)nc(-c5ccccc5)n4)cc3)nc(-c3ccccc3)n2)cc1]

Compound E2 was prepared in the same manner as in Preparation Example 1, except that 2,4-diphenyl-6-(4′-(4,4,5,5-tetramethyl-1,3,2-dioxaborolan-2-yl)-[1,1′-biphenyl]-2-yl)pyrimidine was used instead of the 2,4-diphenyl-6-(4′-(4,4,5,5-tetramethyl-1,3,2-dioxaborolan-2-yl)-[1,1′-biphenyl]-4-yl)pyrimidine in Preparation Example 1.

MS [M+H]⁺=741

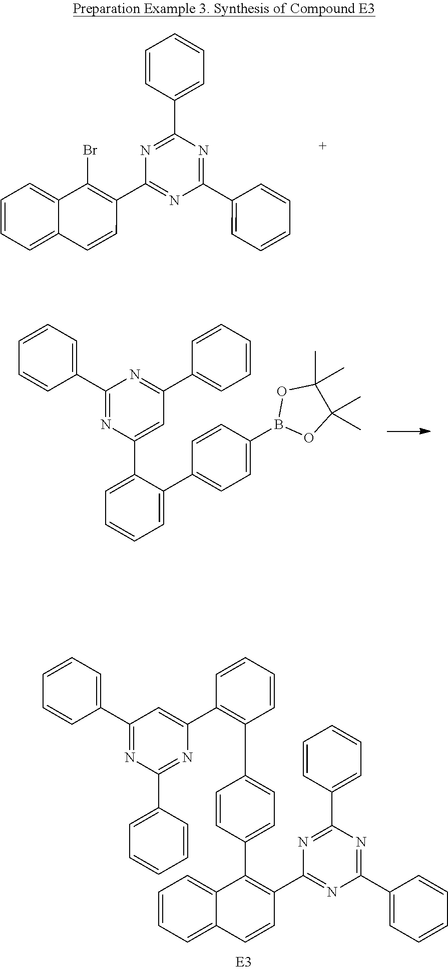 [SMILES: Brc1c(-c2nc(-c3ccccc3)nc(-c3ccccc3)n2)ccc2ccccc12 ; CC1(C)OB(c2ccc(-c3ccccc3-c3cc(-c4ccccc4)nc(-c4ccccc4)n3)cc2)OC1(C)C ; c1ccc(-c2cc(-c3ccccc3-c3ccc(-c4c(-c5nc(-c6ccccc6)nc(-c6ccccc6)n5)ccc5ccccc45)cc3)nc(-c3ccccc3)n2)cc1]

Compound E3 was prepared in the same manner as in Preparation Example 2, except that 2-(1-bromonaphthalen-2-yl)-4,6-diphenyl-1,3,5-triazine was used instead of the 2-(2-bromonaphthalen-1-yl)-4,6-diphenyl-1,3,5-triazine in Preparation Example 2.

MS [M+H]⁺=741

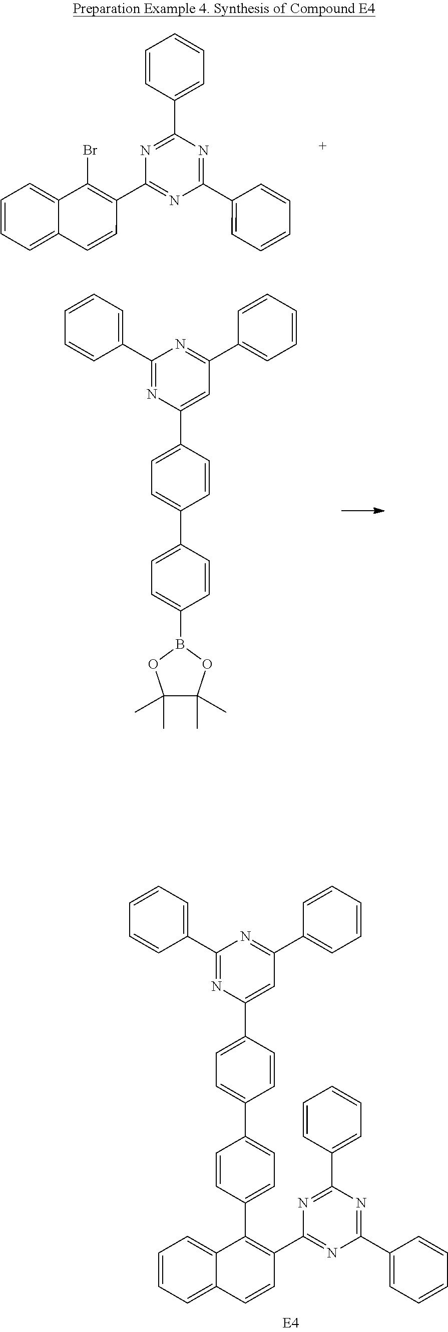

Compound E4 was prepared in the same manner as in Preparation Example 3, except that 2,4-diphenyl-6-(4′-(4,4,5,5-tetramethyl-1,3,2-dioxaborolan-2-yl)-[1,1′-biphenyl]-4-yl)pyrimidine was used instead of a compound 2,4-diphenyl-6-(4′-(4,4,5,5-tetramethyl-1,3,2-dioxaborolan-2-yl)-[1,1′-biphenyl]-2-yl)pyrimidine in Preparation Example 3.

MS[M+H]⁺=741

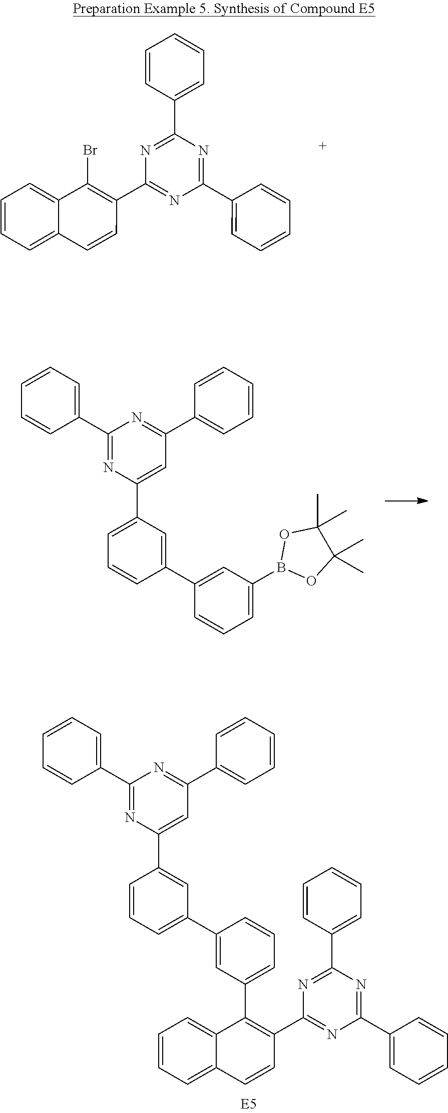 [SMILES: Brc1c(-c2nc(-c3ccccc3)nc(-c3ccccc3)n2)ccc2ccccc12 ; CC1(C)OB(c2cccc(-c3cccc(-c4cc(-c5ccccc5)nc(-c5ccccc5)n4)c3)c2)OC1(C)C ; c1ccc(-c2cc(-c3cccc(-c4cccc(-c5c(-c6nc(-c7ccccc7)nc(-c7ccccc7)n6)ccc6ccccc56)c4)c3)nc(-c3ccccc3)n2)cc1]

Compound E5 was prepared in the same manner as in Preparation Example 3, except that 2,4-diphenyl-6-(3′-(4,4,5,5-tetramethyl-1,3,2-dioxaborolan-2-yl)-[1,1′-biphenyl]-3-yl)pyrimidine was used instead of a compound 2,4-diphenyl-6-(4′-(4,4,5,5-tetramethyl-1,3,2-dioxaborolan-2-yl)-[1,1′-biphenyl]-2-yl)pyrimidine in Preparation Example 3.

MS[M+H]⁺=741

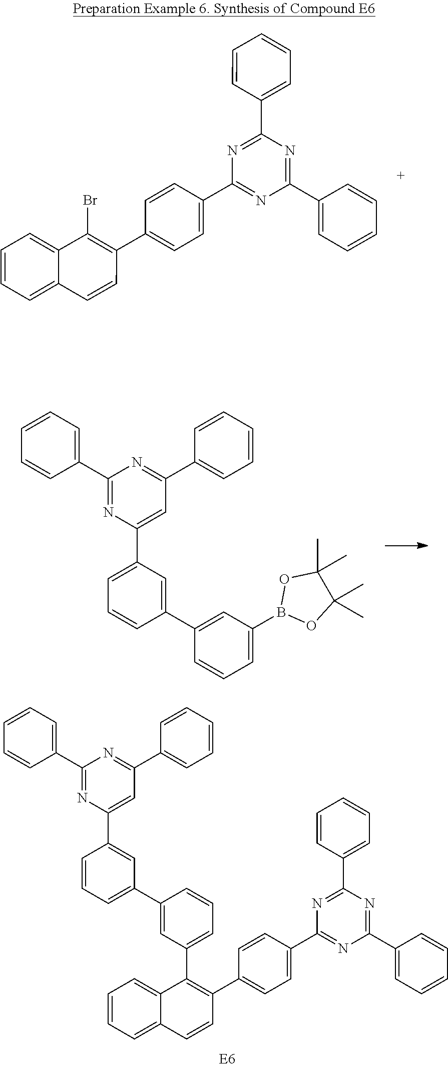

Compound E6 was prepared in the same manner as in Preparation Example 5, except that 2-(4-(1-bromonaphthalen-2-yl)phenyl)-4,6-diphenyl-1,3,5-triazine was used instead of a compound 2-(1-bromonaphthalen-2-yl)-4,6-diphenyl-1,3,5-triazine in Preparation Example 5.

MS[M+H]⁺=818

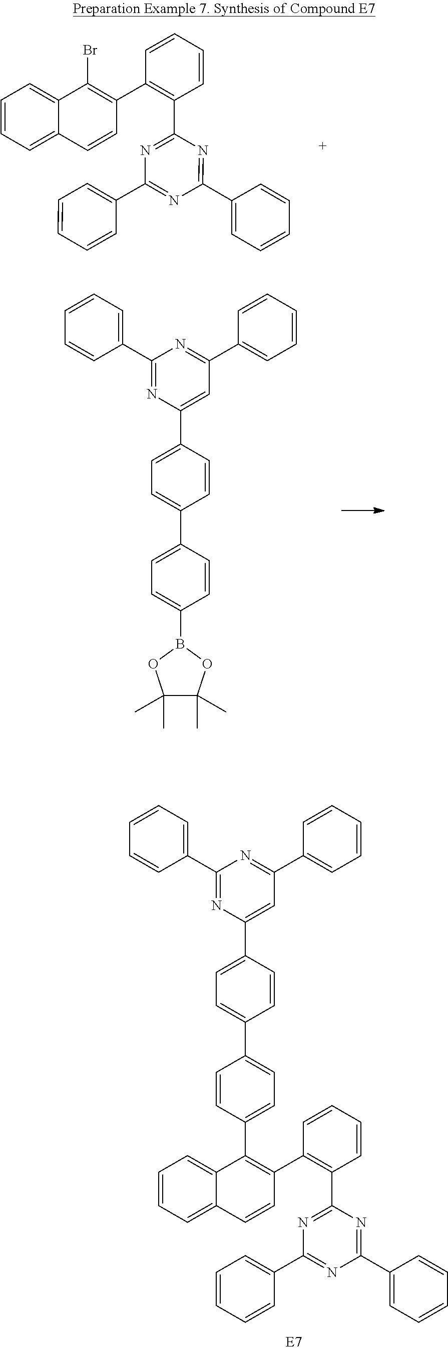

Compound E7 was prepared in the same manner as in Preparation Example 4, except that 2-(2-(1-bromonaphthalen-2-yl)phenyl)-4,6-diphenyl-1,3,5-triazine was used instead of 2-(1-bromonaphthalen-2-yl)-4,6-diphenyl-1,3,5-triazine in Preparation Example 4.

MS[M+H]⁺=818

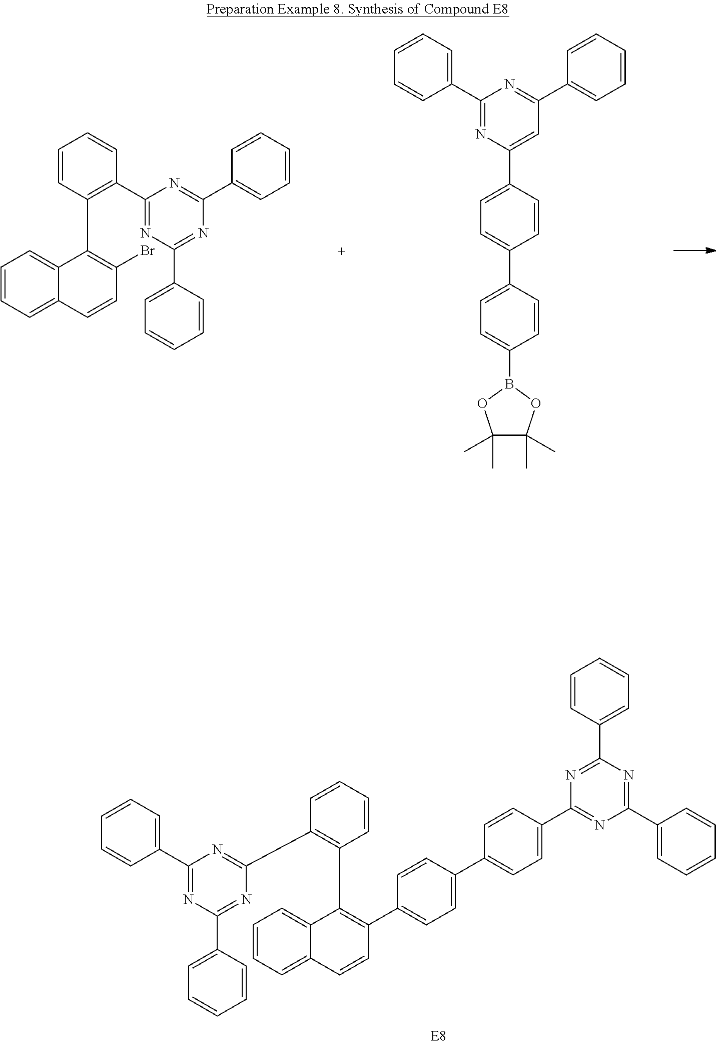 [SMILES: Brc1ccc2ccccc2c1-c1ccccc1-c1nc(-c2ccccc2)nc(-c2ccccc2)n1 ; CC1(C)OB(c2ccc(-c3ccc(-c4cc(-c5ccccc5)nc(-c5ccccc5)n4)cc3)cc2)OC1(C)C ; c1ccc(-c2nc(-c3ccccc3)nc(-c3ccc(-c4ccc(-c5ccc6ccccc6c5-c5ccccc5-c5nc(-c6ccccc6)nc(-c6ccccc6)n5)cc4)cc3)n2)cc1]

Compound E8 was prepared in the same manner as in Preparation Example 4, except that 2-(2-(2-bromonaphthalen-1-yl)phenyl)-4,6-diphenyl-1,3,5-triazine was used instead of the compound 2-(1-bromonaphthalen-2-yl)-4,6-diphenyl-1,3,5-triazine in Preparation Example 4.

MS[M+H]⁺=818

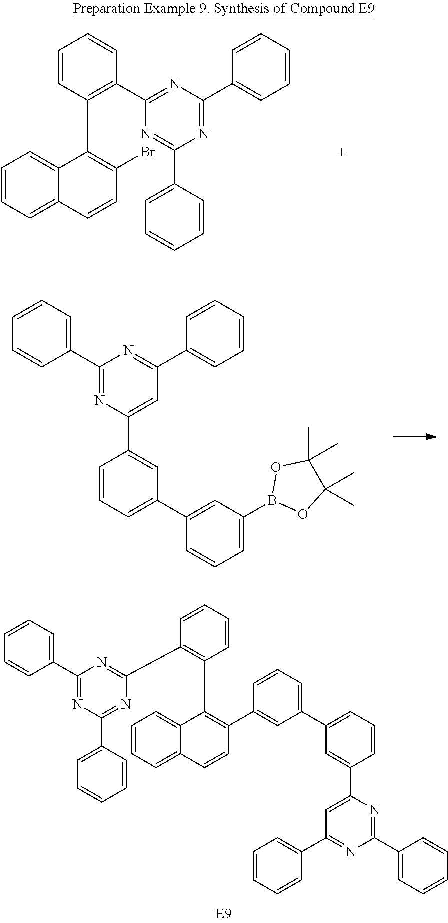

Compound E9 was prepared in the same manner as in Preparation Example 8, except that 2,4-diphenyl-6-(3′-(4,4,5,5-tetramethyl-1,3,2-dioxaborolan-2-yl)-[1,1′-biphenyl]-3-yl)pyrimidine was used instead of the compound 2,4-diphenyl-6-(4′-(4,4,5,5-tetramethyl-1,3,2-dioxaborolan-2-yl)-[1,1′-biphenyl]-4-yl)pyrimidine in Preparation Example 8.

MS [M+H]⁺=818

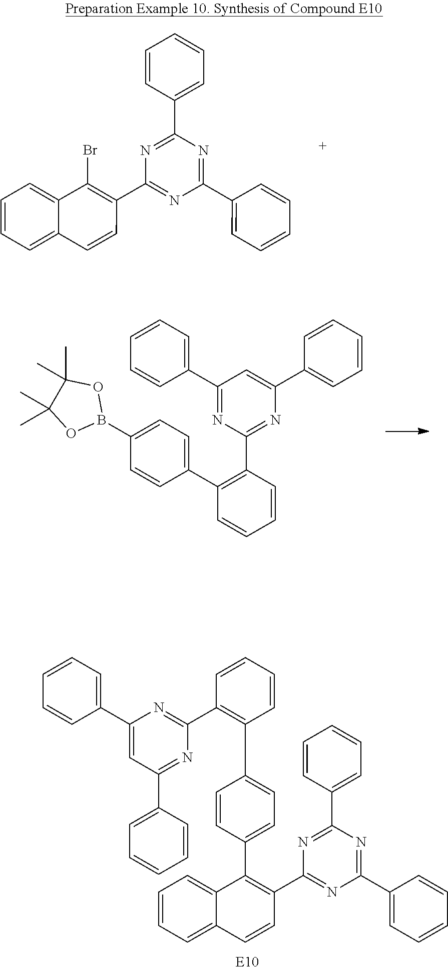 [SMILES: Brc1c(-c2nc(-c3ccccc3)nc(-c3ccccc3)n2)ccc2ccccc12 ; CC1(C)OB(c2ccc(-c3ccccc3-c3nc(-c4ccccc4)cc(-c4ccccc4)n3)cc2)OC1(C)C ; c1ccc(-c2cc(-c3ccccc3)nc(-c3ccccc3-c3ccc(-c4c(-c5nc(-c6ccccc6)nc(-c6ccccc6)n5)ccc5ccccc45)cc3)n2)cc1]

Compound E10 was prepared in the same manner as in Preparation Example 3, except that 4,6-diphenyl-2-(4′-(4,4,5,5-tetramethyl-1,3,2-dioxaborolan-2-yl)-[1,1′-biphenyl]-2-yl)pyrimidine was used instead of the compound 2,4-diphenyl-6-(4′-(4,4,5,5-tetramethyl-1,3,2-dioxaborolan-2-yl)-[1,1′-biphenyl]-2-yl)pyrimidine in Preparation Example 3.

MS[M+H]⁺=741

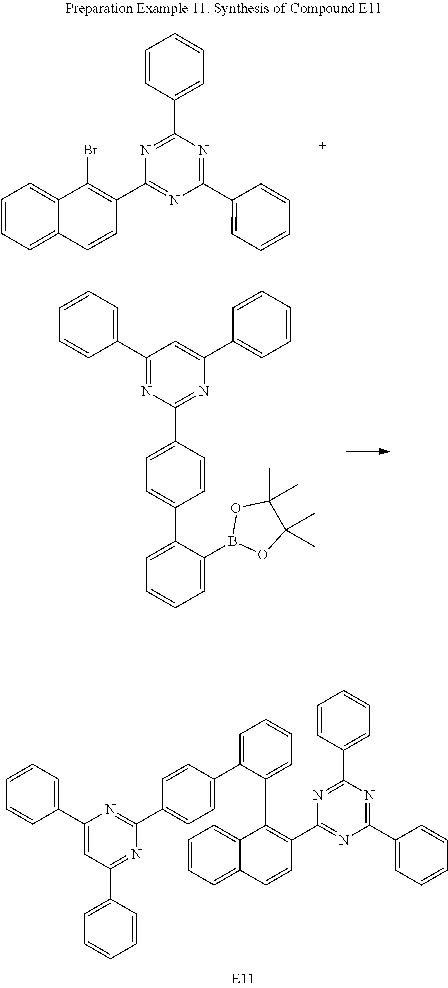

Compound E11 was prepared in the same manner as in Preparation Example 3, except that 4,6-diphenyl-2-(2′-(4,4,5,5-tetramethyl-1,3,2-dioxaborolan-2-yl)-[1,1′-biphenyl]-4-yl)pyrimidine was used instead of the compound 2,4-diphenyl-6-(4′-(4,4,5,5-tetramethyl-1,3,2-dioxaborolan-2-yl)-[1,1′-biphenyl]-2-yl)pyrimidine in Preparation Example 3.

MS[M+H]⁺=741

### Example 1-1

A glass substrate thinly coated with indium tin oxide (ITO) to have a thickness of 1,000 Å was put into distilled water in which a detergent was dissolved, and ultrasonically washed. In this case, a product manufactured by the Fischer Co., was used as the detergent, and distilled water twice filtered using a filter manufactured by Millipore Co., was used as the distilled water. After the ITO was washed for 30 minutes, ultrasonic washing was repeated twice by using distilled water for 10 minutes. After the washing using distilled water was completed, ultrasonic washing was conducted by using isopropyl alcohol, acetone, and methanol solvents, and the resulting product was dried and then transported to a plasma washing machine. Furthermore, the substrate was cleaned by using oxygen plasma for 5 minutes, and then was transported to a vacuum deposition machine.

Compounds of the following Compound HI1 and the following Compound HI2 were thermally vacuum deposited to have a thickness of 100 Å at a ratio of 98:2 (molar ratio) on a transparent ITO electrode, which is the positive electrode thus prepared, thereby forming a hole injection layer. A compound of the following Chemical Formula HT1 (1,150 Å) was vacuum deposited on the hole injection layer, thereby forming a hole transport layer. Subsequently, a compound of the following Chemical Formula EB1 was vacuum deposited to have a film thickness of 50 Å on the hole transport layer, thereby forming an electron blocking layer. Subsequently, a compound of the following Chemical Formula BH and a compound of the following Chemical Formula BD were vacuum deposited at a weight ratio of 50:1 to have a film thickness of 200 Å on the electron blocking layer, thereby forming a light emitting layer. A compound of the following Chemical Formula HB1 was vacuum deposited to have a film thickness of 50 Å on the light emitting layer, thereby forming a hole blocking layer. Subsequently, Compound E1 synthesized in Preparation Example 1 and a compound of the following Chemical Formula LiQ were vacuum deposited at a weight ratio of 1:1 on the hole blocking layer, thereby forming an electron injection and transport layer having a thickness of 30 Å. Lithium fluoride (LiF) and aluminum were subsequently deposited to have a thickness of 12 Å and 1,000 Å, respectively, on the electron injection and transport layer, thereby forming a negative electrode.

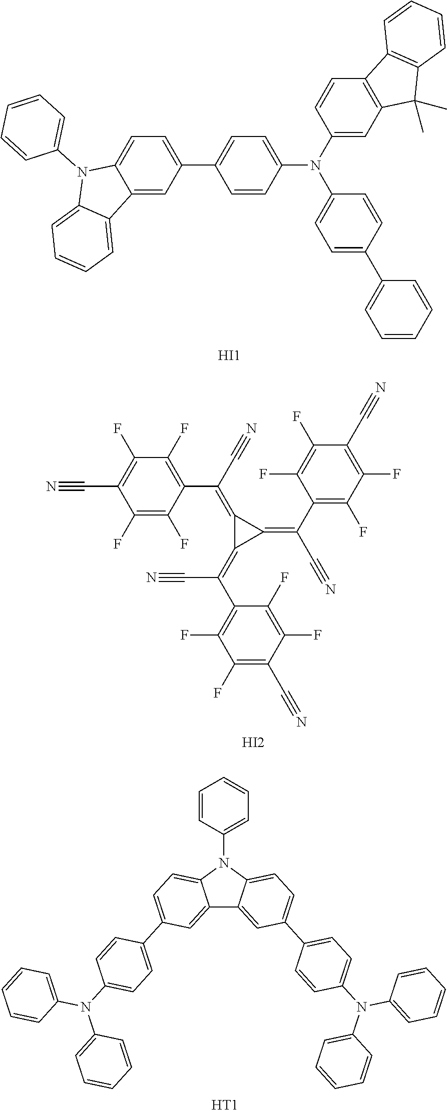

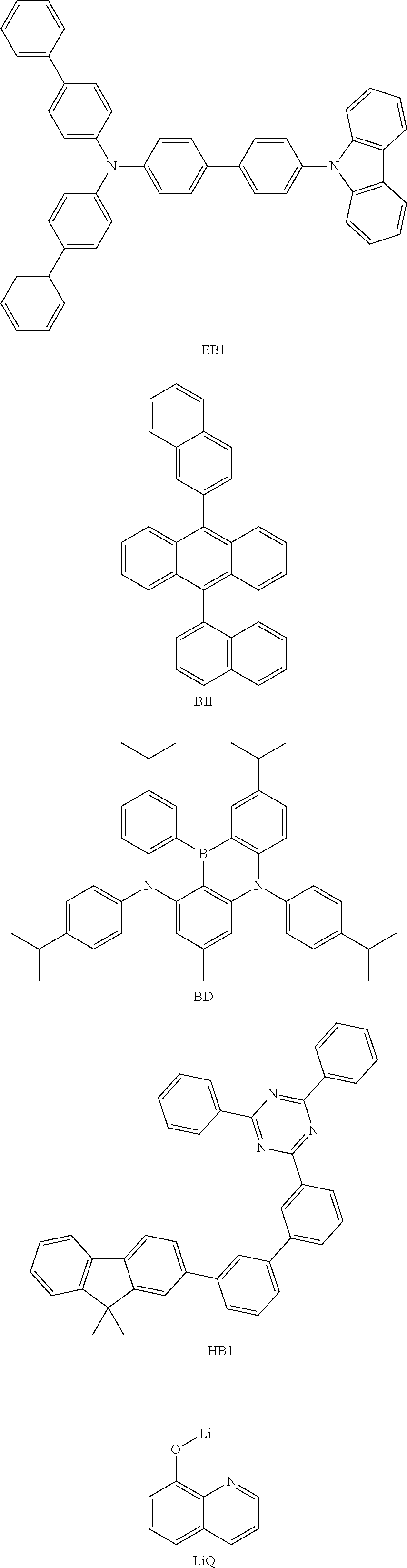

In the aforementioned procedure, the deposition rate of the organic material was maintained at 0.4 to 0.7 Å/sec, the deposition rates of lithium fluoride and aluminum of the negative electrode were maintained at 0.3 Å/sec and at 2 Å/sec, respectively, and the degree of vacuum during the deposition was maintained at 2×10⁻⁷ to 5×10⁻⁶ torr, thereby manufacturing an organic light emitting device.

### Examples 1-2 to 1-11

Organic light emitting devices were manufactured in the same manner as in Example 1-1, except that the compounds described in the following Table 1 were used instead of Compound E1 in Example 1-1.

### Comparative Examples 1-1 to 1-10

Organic light emitting devices were manufactured in the same manner as in Example 1-1, except that the compounds described in the following Table 1 were used instead of Compound E1 in Example 1-1. The compounds of ET-1, ET-2, ET-3, ET-4, ET-5, ET-6, ET-7, ET-8, ET-9 and ET-10 used in the following Table 1 are as follows.

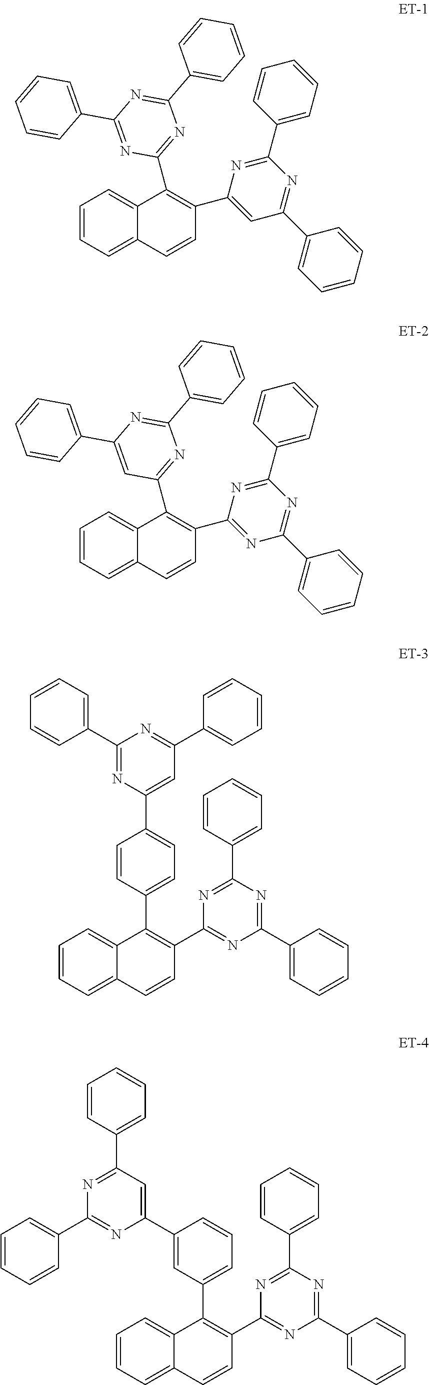 [SMILES: c1ccc(-c2cc(-c3ccc4ccccc4c3-c3nc(-c4ccccc4)nc(-c4ccccc4)n3)nc(-c3ccccc3)n2)cc1 ; c1ccc(-c2cc(-c3c(-c4nc(-c5ccccc5)nc(-c5ccccc5)n4)ccc4ccccc34)nc(-c3ccccc3)n2)cc1 ; c1ccc(-c2cc(-c3ccc(-c4c(-c5nc(-c6ccccc6)nc(-c6ccccc6)n5)ccc5ccccc45)cc3)nc(-c3ccccc3)n2)cc1 ; c1ccc(-c2cc(-c3cccc(-c4c(-c5nc(-c6ccccc6)nc(-c6ccccc6)n5)ccc5ccccc45)c3)nc(-c3ccccc3)n2)cc1]

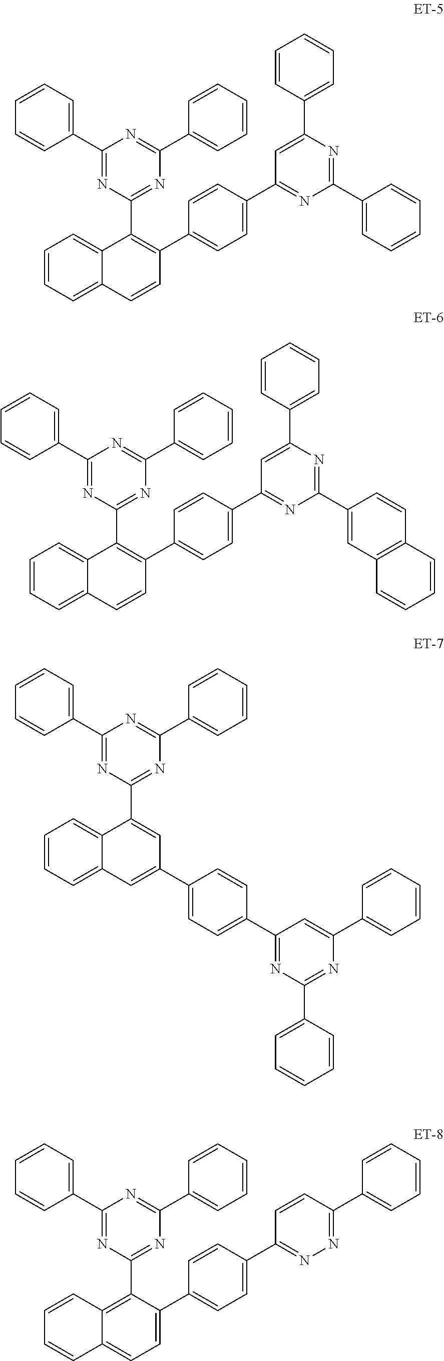 [SMILES: c1ccc(-c2cc(-c3ccc(-c4ccc5ccccc5c4-c4nc(-c5ccccc5)nc(-c5ccccc5)n4)cc3)nc(-c3ccccc3)n2)cc1 ; c1ccc(-c2cc(-c3ccc(-c4ccc5ccccc5c4-c4nc(-c5ccccc5)nc(-c5ccccc5)n4)cc3)nc(-c3ccc4ccccc4c3)n2)cc1 ; c1ccc(-c2cc(-c3ccc(-c4cc(-c5nc(-c6ccccc6)nc(-c6ccccc6)n5)c5ccccc5c4)cc3)nc(-c3ccccc3)n2)cc1 ; c1ccc(-c2ccc(-c3ccc(-c4ccc5ccccc5c4-c4nc(-c5ccccc5)nc(-c5ccccc5)n4)cc3)nn2)cc1]

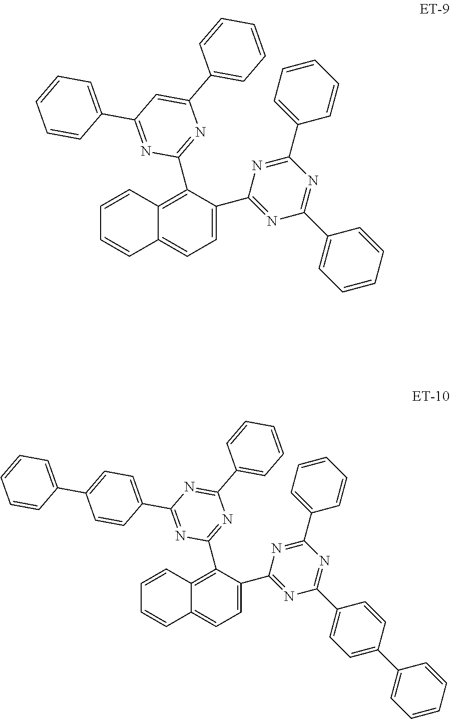 [SMILES: c1ccc(-c2cc(-c3ccccc3)nc(-c3c(-c4nc(-c5ccccc5)nc(-c5ccccc5)n4)ccc4ccccc34)n2)cc1 ; c1ccc(-c2ccc(-c3nc(-c4ccccc4)nc(-c4ccc5ccccc5c4-c4nc(-c5ccccc5)nc(-c5ccc(-c6ccccc6)cc5)n4)n3)cc2)cc1]

When current was applied to the organic light emitting devices manufactured in Examples 1-1 to 1-11 and Comparative Examples 1-1 to 1-10, the voltages, efficiencies, color coordinates, and service lives were measured, and the results are shown in the following [Table 1]. T95 means the time taken for the luminance to be reduced to 95% of the initial luminance (1,600 nit).

TABLE 1

| | Compound | | | | |
|---|---|---|---|---|---|
| | (Electron | | | | |
| | injection | | | | |
| | and | | | Color | |
| | transport | voltage | efficiency | coordinate | T95 |
| | layer) | (V@20 mA/cm²) | (cd/A@20 mA/cm²) | (x, y) | (hr) |
| Example 1-1 | Compound | 4.25 | 6.03 | (0.140, | 236 |
| | E1 | | | 0.041) | |
| Example 1-2 | Compound | 4.43 | 6.22 | (0.140, | 228 |
| | E2 | | | 0.041) | |
| Example 1-3 | Compound | 4.31 | 6.31 | (0.141, | 245 |
| | E3 | | | 0.041) | |
| Example 1-4 | Compound | 4.12 | 6.44 | (0.141, | 269 |
| | E4 | | | 0.040) | |
| Example 1-5 | Compound | 4.25 | 6.26 | (0.140, | 239 |
| | E5 | | | 0.041) | |
| Example 1-6 | Compound | 4.36 | 6.31 | (0.141, | 257 |
| | E6 | | | 0.040) | |
| Example 1-7 | Compound | 4.28 | 6.47 | (0.140, | 231 |
| | E7 | | | 0.040) | |
| Example 1-8 | Compound | 4.41 | 6.10 | (0.140, | 264 |
| | E8 | | | 0.040) | |
| Example 1-9 | Compound | 4.30 | 6.19 | (0.141, | 227 |
| | E9 | | | 0.041) | |
| Example 1- | Compound | 4.27 | 6.18 | (0.139, | 252 |
| 10 | E10 | | | 0.041) | |
| Example 1- | Compound | 4.33 | 6.27 | (0.140, | 248 |
| 11 | E11 | | | 0.041) | |
| Comparative | ET-1 | 4.58 | 5.34 | (0.141, | 78 |
| Example 1-1 | | | | 0.042) | |
| Comparative | ET-2 | 4.61 | 4.93 | (0.139, | 81 |
| Example 1-2 | | | | 0.041) | |
| Comparative | ET-3 | 5.17 | 4.24 | (0.140, | 185 |
| Example 1-3 | | | | 0.040) | |
| Comparative | ET-4 | 5.01 | 4.12 | (0.141, | 179 |
| Example 1-4 | | | | 0.041) | |
| Comparative | ET-5 | 5.65 | 4.38 | (0.139, | 175 |
| Example 1-5 | | | | 0.040) | |
| Comparative | ET-6 | 6.42 | 4.58 | (0.141, | 197 |
| Example 1-6 | | | | 0.040) | |
| Comparative | ET-7 | 4.87 | 5.21 | (0.140, | 201 |
| Example 1-7 | | | | 0.039) | |
| Comparative | ET-8 | 5.24 | 5.14 | (0.139, | 210 |
| Example 1-8 | | | | 0.039) | |
| Comparative | ET-9 | 5.19 | 5.29 | (0.140, | 206 |
| Example 1-9 | | | | 0.038) | |
| Comparative | ET-10 | 4.69 | 5.32 | (0.140, | 185 |
| Example 1- | | | | 0.039) | |
| 10 | | | | | |

As seen in Table 1, the organic light emitting device manufactured by using the compound of the present invention as an electron injection and transport layer exhibits excellent characteristics in terms of efficiency, driving voltage, and/or stability of the organic light emitting device.

Since the compounds of the present invention have a longer linker length between the N-containing ring group and naphthalene than ET-1 to ET-10 in Comparative Examples 1-1 to 1-10, it can be seen that Experimental Examples 1-1 to 1-11 have lower driving voltages, higher efficiency, and longer service lives (T95) than those of Comparative Examples 1-1 to 1-10.

That is, through Examples 1-1 to 1-11, it could be confirmed that when an electron injection and transport layer, in which the conjugation is appropriately adjusted by changing the linker length between the N-containing ring group and naphthalene, is used rather than an electron injection and transport layer generally used, the properties of low voltage, high efficiency and long service life are exhibited in terms of properties as an electron transport layer.

Specifically, it could be confirmed that the compound of the present invention had higher efficiency and longer service life (T95) than those in ET-1, ET-2, ET-9 and ET-10 of Comparative Examples 1-1, 1-2, 1-9 and 1-10 in which the N-containing ring group and naphthalene are directly bonded, exhibited properties of low voltage, high efficiency and long service life compared to ET-3 to ET-6 of Comparative Examples 1-3 to 1-6 in which the N-containing ring group and naphthalene are linked via phenylene, was more efficient than ET-7 in Comparative Examples 1-7 in which the N-containing ring group and triazine are bonded to positions 1 and 3 of naphthalene instead of positions 1 and 2 of naphthalene, and exhibited lower driving voltage and higher efficiency than those of ET-8 of Comparative Example 1-8 which includes an N-containing ring group including two N's at positions different from those of X1 to X3 of Chemical Formula 1 of the present application.

Although the preferred exemplary embodiments (an electron injection and transport layer) of the present invention have been described above, the present invention is not limited thereto, and various modifications can be made and carried out within the scope of the claims and the detailed description of the invention, and also fall within the scope of the invention.

1. A compound of Chemical Formula 1: 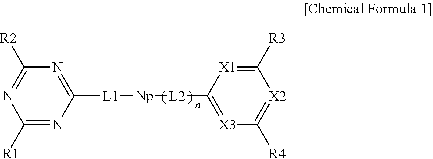
wherein in Chemical Formula 1:
L1 is a direct bond or a substituted or unsubstituted arylene group;
L2 is a substituted or unsubstituted arylene group;
n is 2 or 3, and the L2s are each the same as or different from each other;
X1 to X3 are the same as or different from each other, and are each independently N or CR′;
two or more of X1 to X3 are N;
R1 to R4 and R′ are the same as or different from each other, and are each independently hydrogen, deuterium, a halogen group, a substituted or unsubstituted alkyl group, a substituted or unsubstituted aryl group, or a substituted or unsubstituted heterocyclic group;
Np is represented by the following Chemical Formula 2-2: 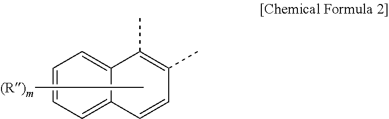
wherein in Chemical Formula 2:
the dotted lines “------” are positions of attachment of Chemical Formula 2 where one is bonded to L1 and the other is bonded to L2 of Chemical Formula 1;
R″ is hydrogen, deuterium, a halogen group, a substituted or unsubstituted alkyl group, a substituted or unsubstituted aryl group, or a substituted or unsubstituted heterocyclic group; and
m is an integer from 0 to 6, and when m is 2 or higher, two or more R″s are the same as or different from each other.

2. The compound of claim 1, wherein Chemical Formula 1 is the following Chemical Formula 1-1 or 1-2: 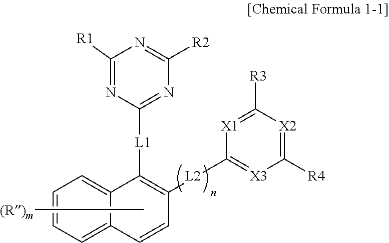 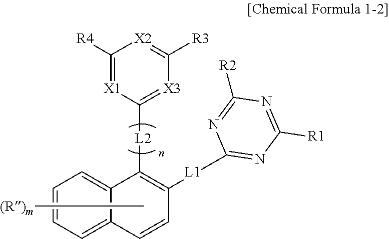
wherein in Chemical Formulae 1-1 and 1-2:
the definitions of L1, L2, n, R1 to R4, X1 to X3, R″ and m are the same as the definitions in Chemical Formula 1.

3. The compound of claim 1, wherein Chemical Formula 1 is the following Chemical Formula 2-1 or 2-2: 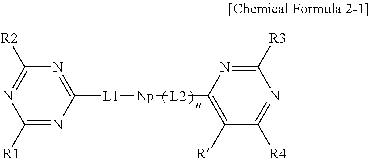 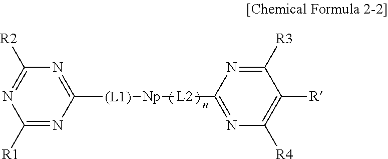
wherein in Chemical Formulae 2-1 and 2-2;
the definitions of L1, L2, n, R1 to R4, R′ and Np are the same as the definitions in Chemical Formula 1.

4. The compound of claim 1, wherein L1 is a direct bond or a substituted or unsubstituted arylene group having 6 to 30 carbon atoms; and
L2 is a substituted or unsubstituted arylene group having 6 to 30 carbon atoms.

5. The compound of claim 1, wherein R1 to R4 are the same as or different from each other, and are each independently a substituted or unsubstituted aryl group having 6 to 30 carbon atoms, or a substituted or unsubstituted heterocyclic group having 2 to 30 carbon atoms.

6. The compound of claim 1, wherein R″ is hydrogen or deuterium.

7. The compound of claim 1, wherein L1 is a direct bond or a substituted or unsubstituted phenylene group; and
L2 is a substituted or unsubstituted phenylene group.

8. The compound of claim 1, wherein R1 to R4 are the same as or different from each other, and are each independently a substituted or unsubstituted phenyl group, a substituted or unsubstituted biphenyl group, a substituted or unsubstituted terphenyl group, a substituted or unsubstituted naphthyl group, or a substituted or unsubstituted fluorenyl group.

9. The compound of claim 1, wherein L1 is a direct bond or an arylene group having 6 to 20 carbon atoms, which is unsubstituted or substituted with deuterium;
L2 is an arylene group having 6 to 20 carbon atoms, which is unsubstituted or substituted with deuterium;
two of X1 to X3 are N; and
R1 to R4 are the same as or different from each other, and are each independently hydrogen, deuterium, or an aryl group having 6 to 20 carbon atoms, which is unsubstituted or substituted with deuterium.

10. The compound of claim 1, wherein the compound of Chemical Formula 1 is any one of the following compounds:

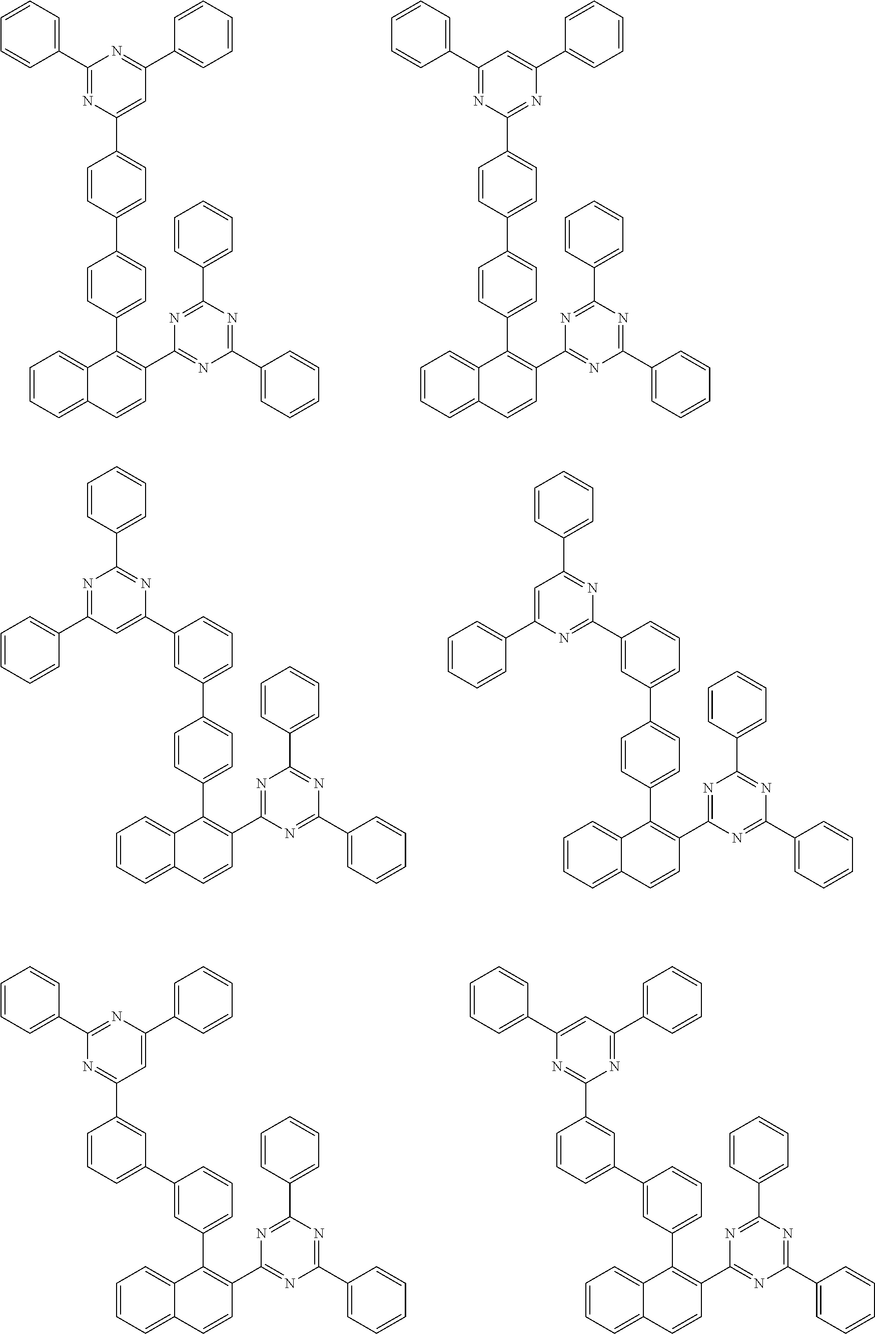

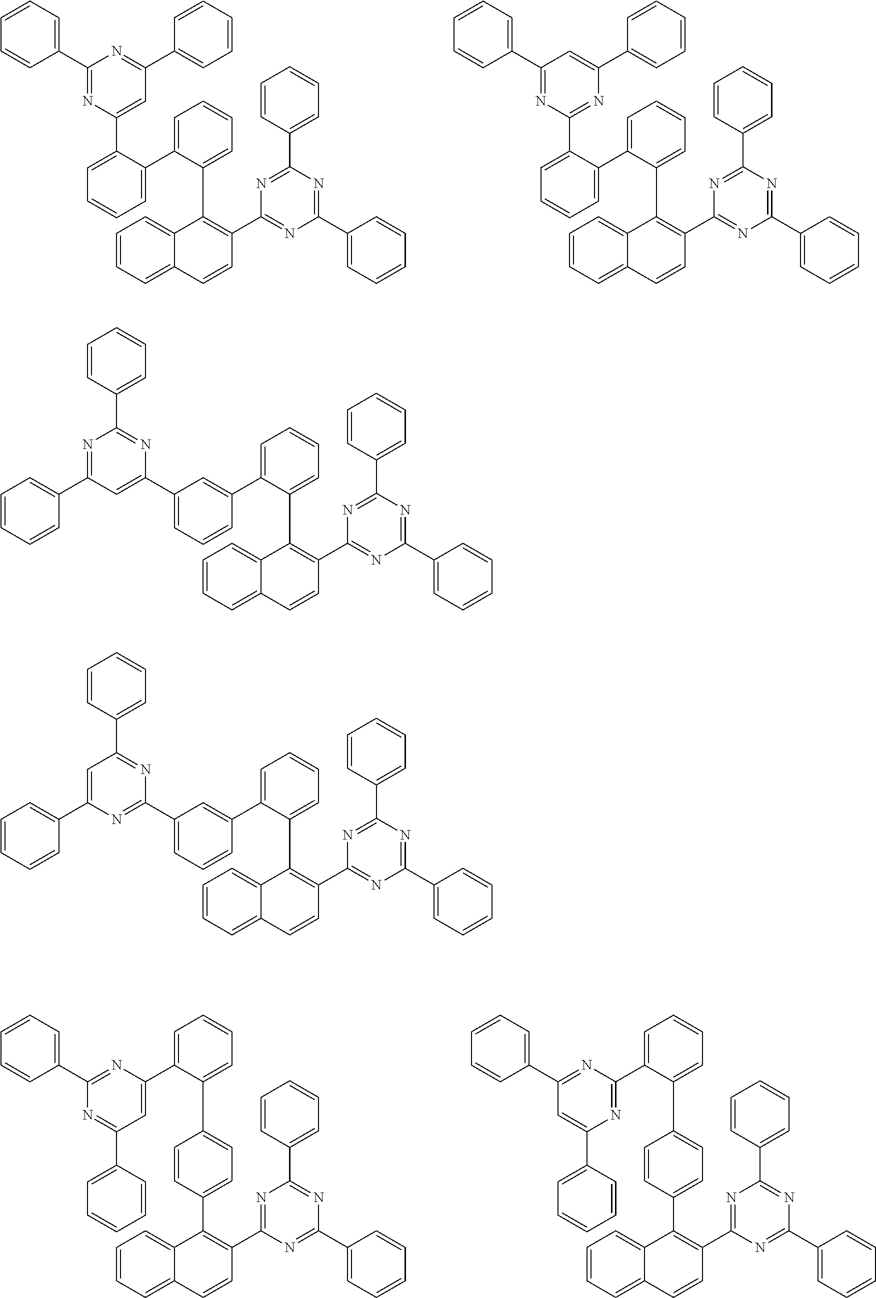

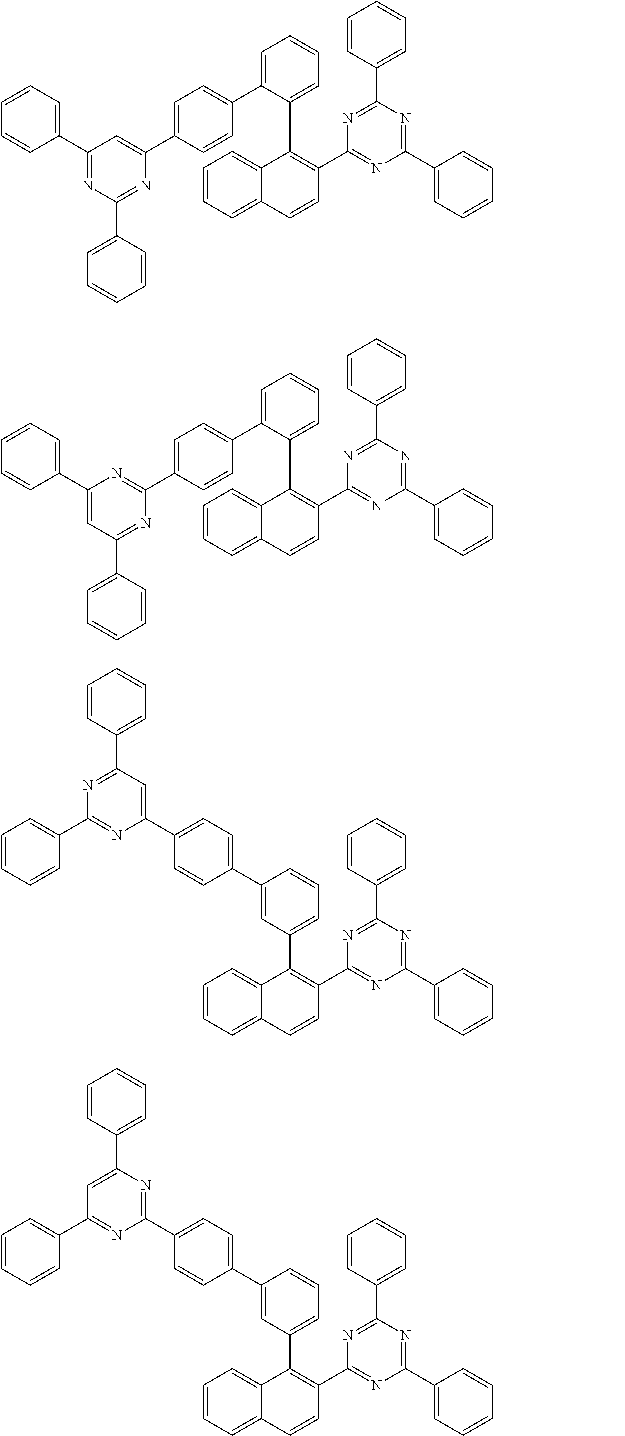

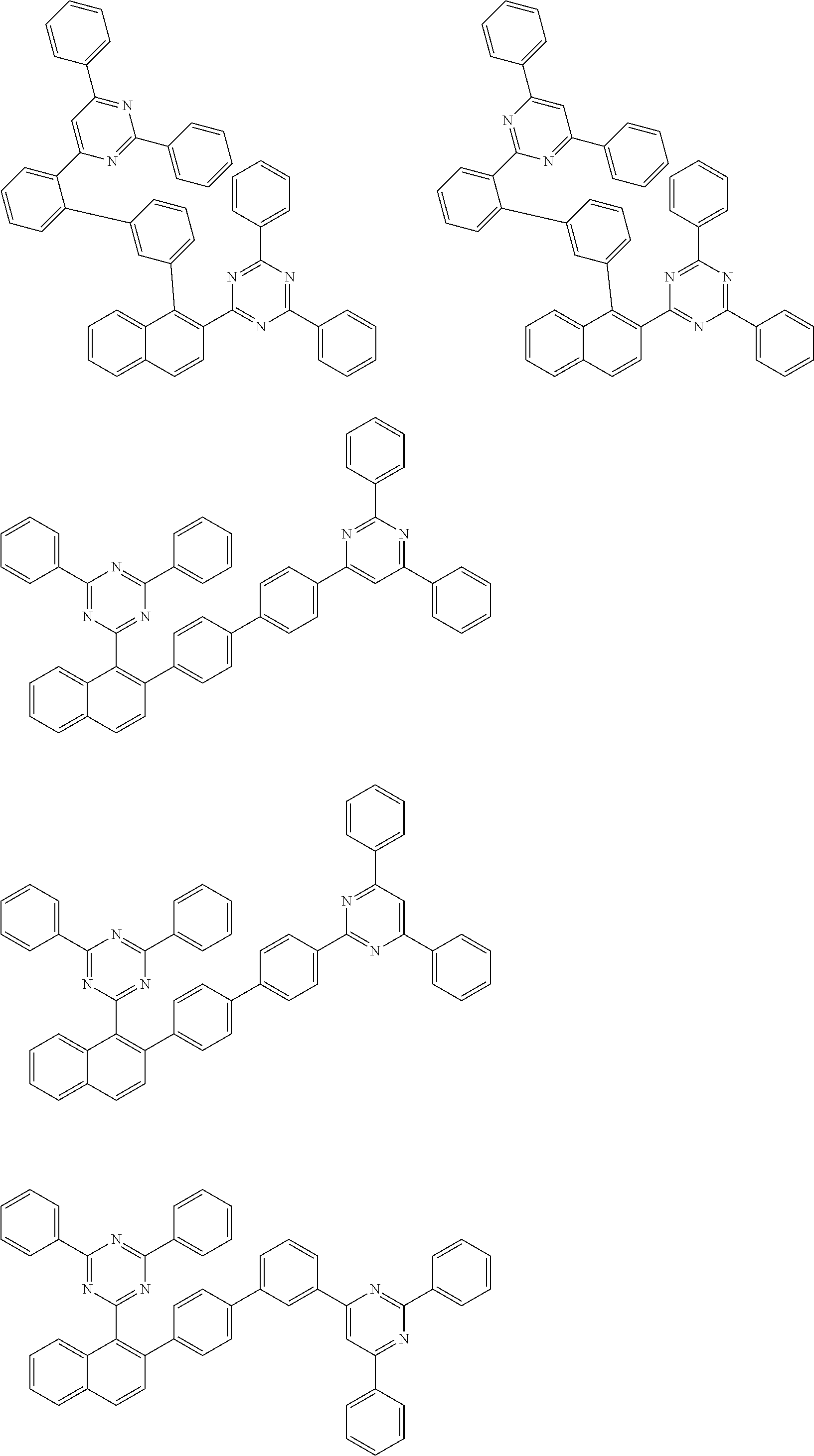

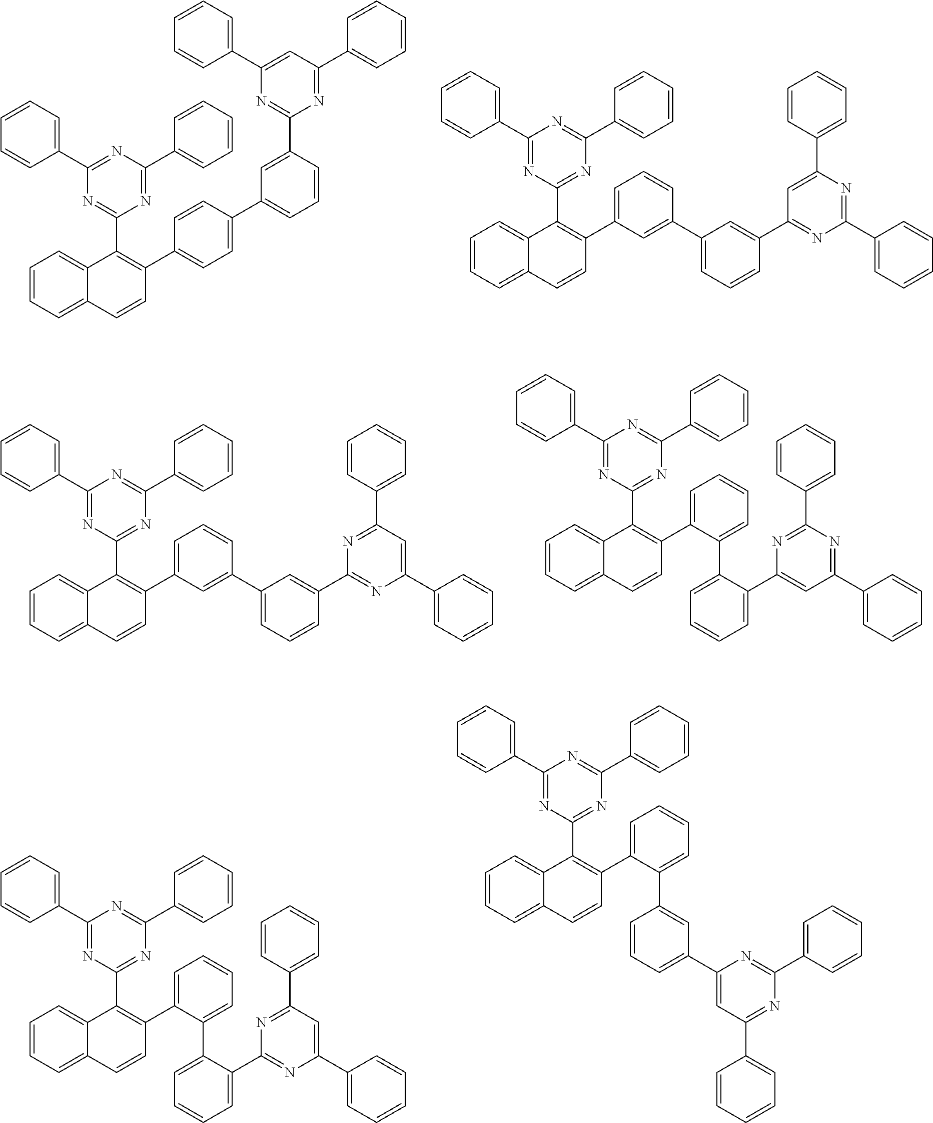

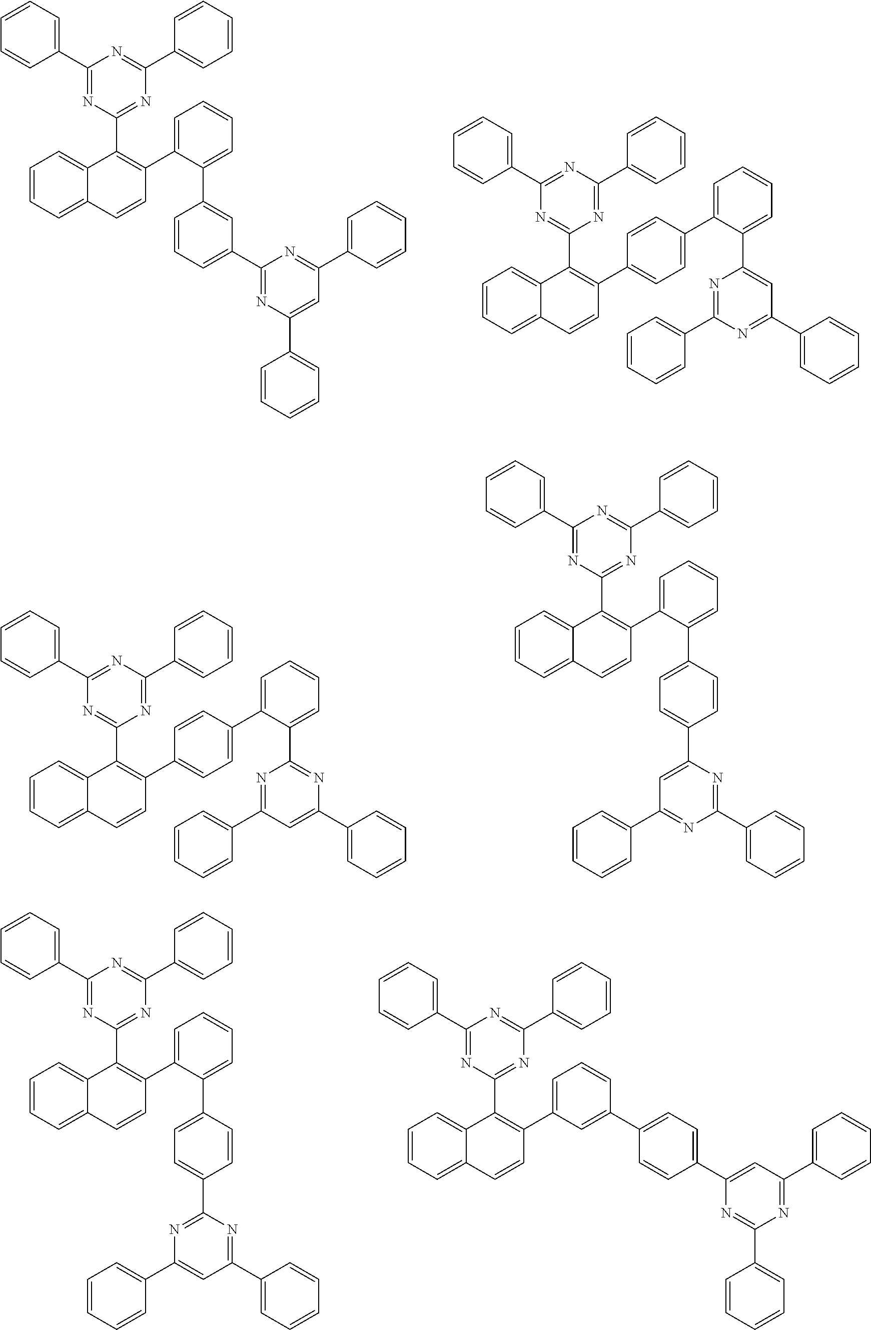

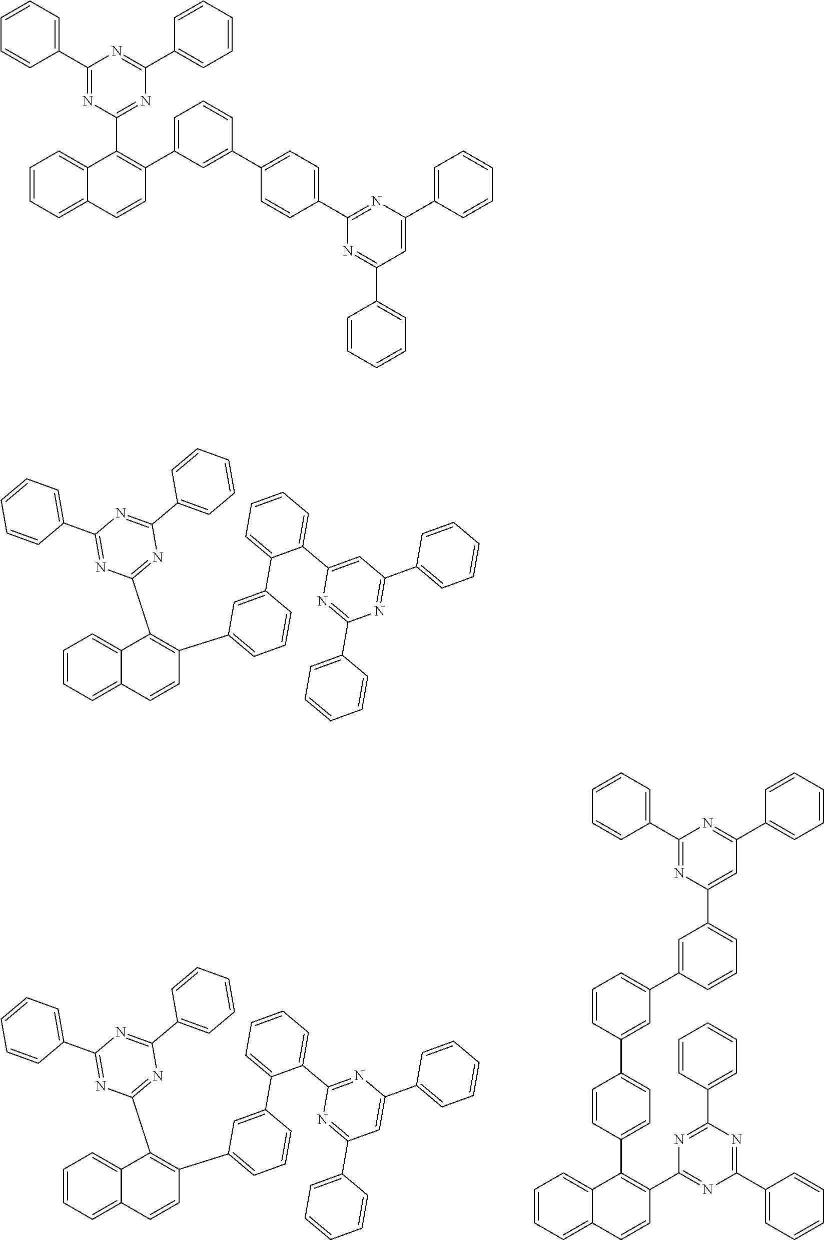 [SMILES: c1ccc(-c2cc(-c3ccccc3)nc(-c3ccc(-c4cccc(-c5ccc6ccccc6c5-c5nc(-c6ccccc6)nc(-c6ccccc6)n5)c4)cc3)n2)cc1 ; c1ccc(-c2cc(-c3ccccc3-c3cccc(-c4ccc5ccccc5c4-c4nc(-c5ccccc5)nc(-c5ccccc5)n4)c3)nc(-c3ccccc3)n2)cc1 ; c1ccc(-c2cc(-c3ccccc3)nc(-c3ccccc3-c3cccc(-c4ccc5ccccc5c4-c4nc(-c5ccccc5)nc(-c5ccccc5)n4)c3)n2)cc1 ; c1ccc(-c2cc(-c3cccc(-c4cccc(-c5ccc(-c6c(-c7nc(-c8ccccc8)nc(-c8ccccc8)n7)ccc7ccccc67)cc5)c4)c3)nc(-c3ccccc3)n2)cc1]

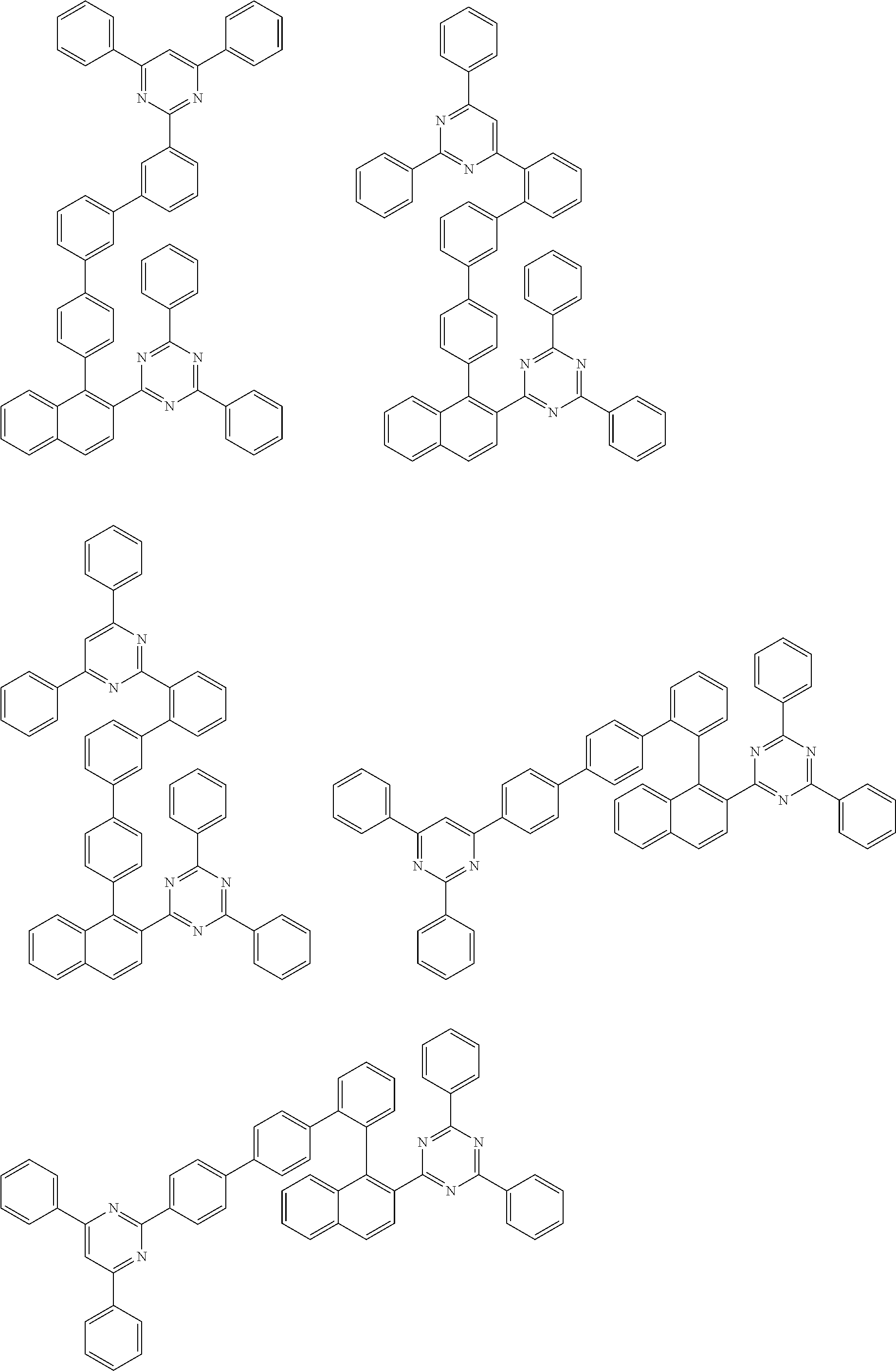

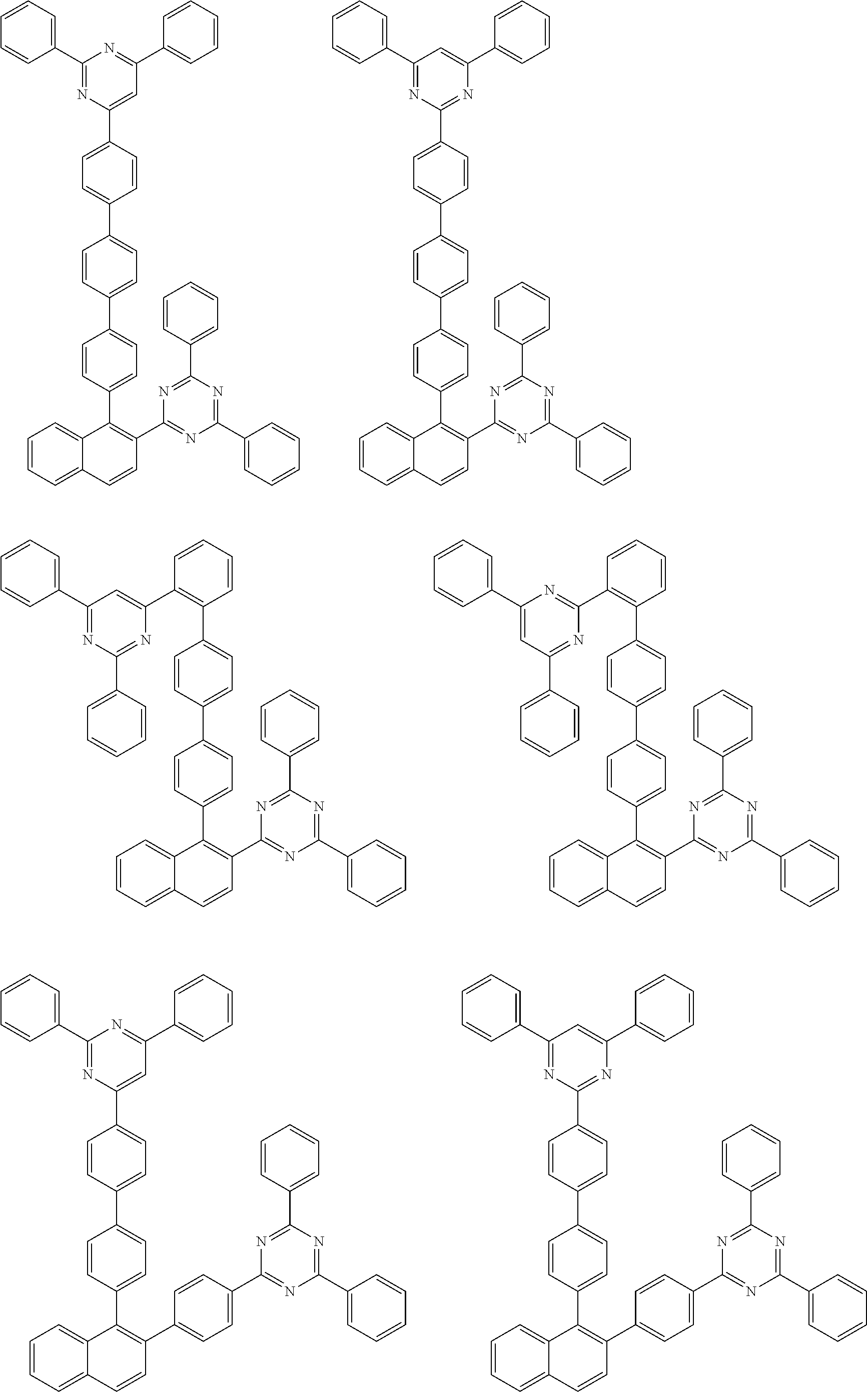

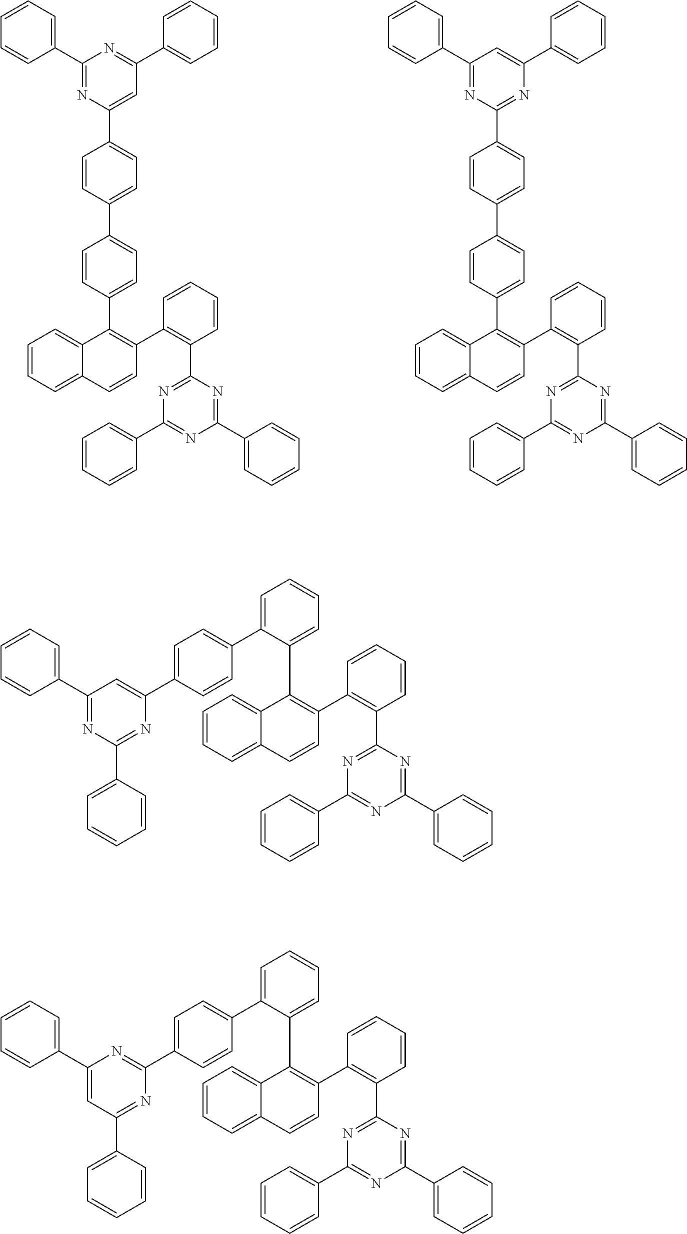 [SMILES: c1ccc(-c2cc(-c3ccc(-c4ccc(-c5c(-c6ccccc6-c6nc(-c7ccccc7)nc(-c7ccccc7)n6)ccc6ccccc56)cc4)cc3)nc(-c3ccccc3)n2)cc1 ; c1ccc(-c2cc(-c3ccccc3)nc(-c3ccc(-c4ccc(-c5c(-c6ccccc6-c6nc(-c7ccccc7)nc(-c7ccccc7)n6)ccc6ccccc56)cc4)cc3)n2)cc1 ; c1ccc(-c2cc(-c3ccc(-c4ccccc4-c4c(-c5ccccc5-c5nc(-c6ccccc6)nc(-c6ccccc6)n5)ccc5ccccc45)cc3)nc(-c3ccccc3)n2)cc1 ; c1ccc(-c2cc(-c3ccccc3)nc(-c3ccc(-c4ccccc4-c4c(-c5ccccc5-c5nc(-c6ccccc6)nc(-c6ccccc6)n5)ccc5ccccc45)cc3)n2)cc1]

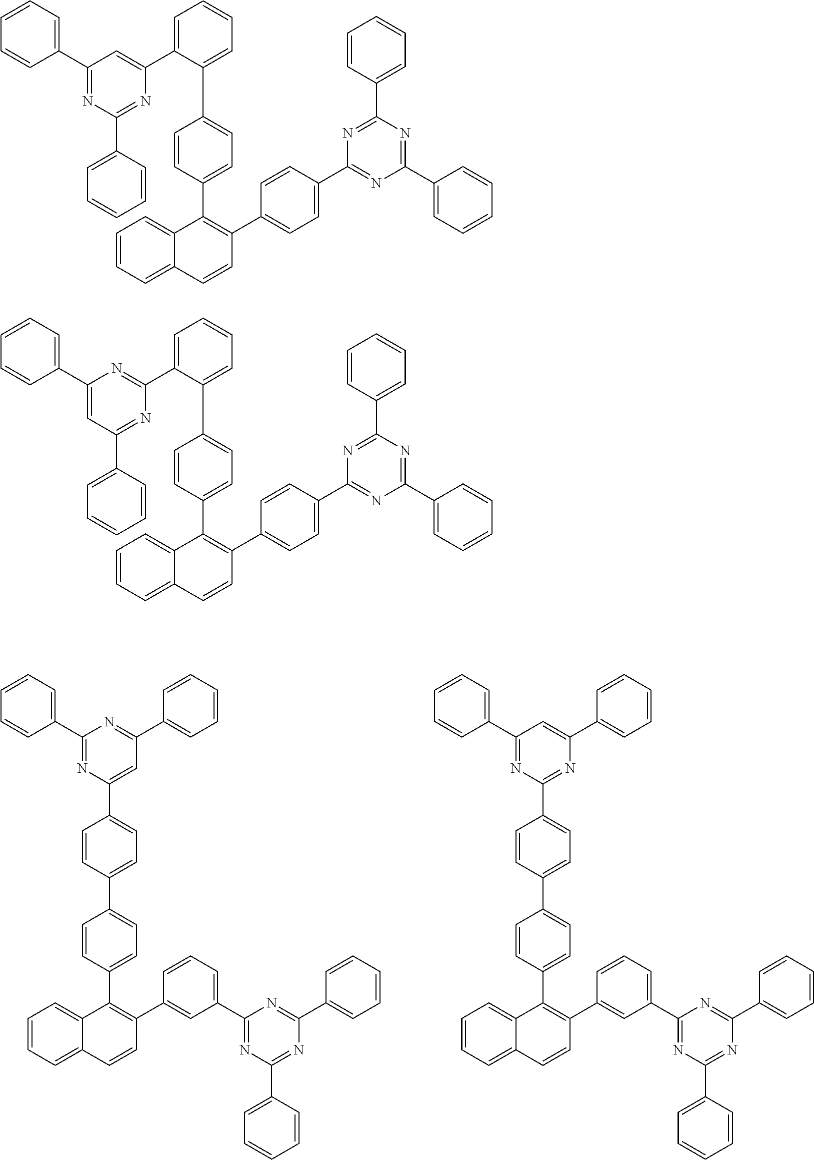

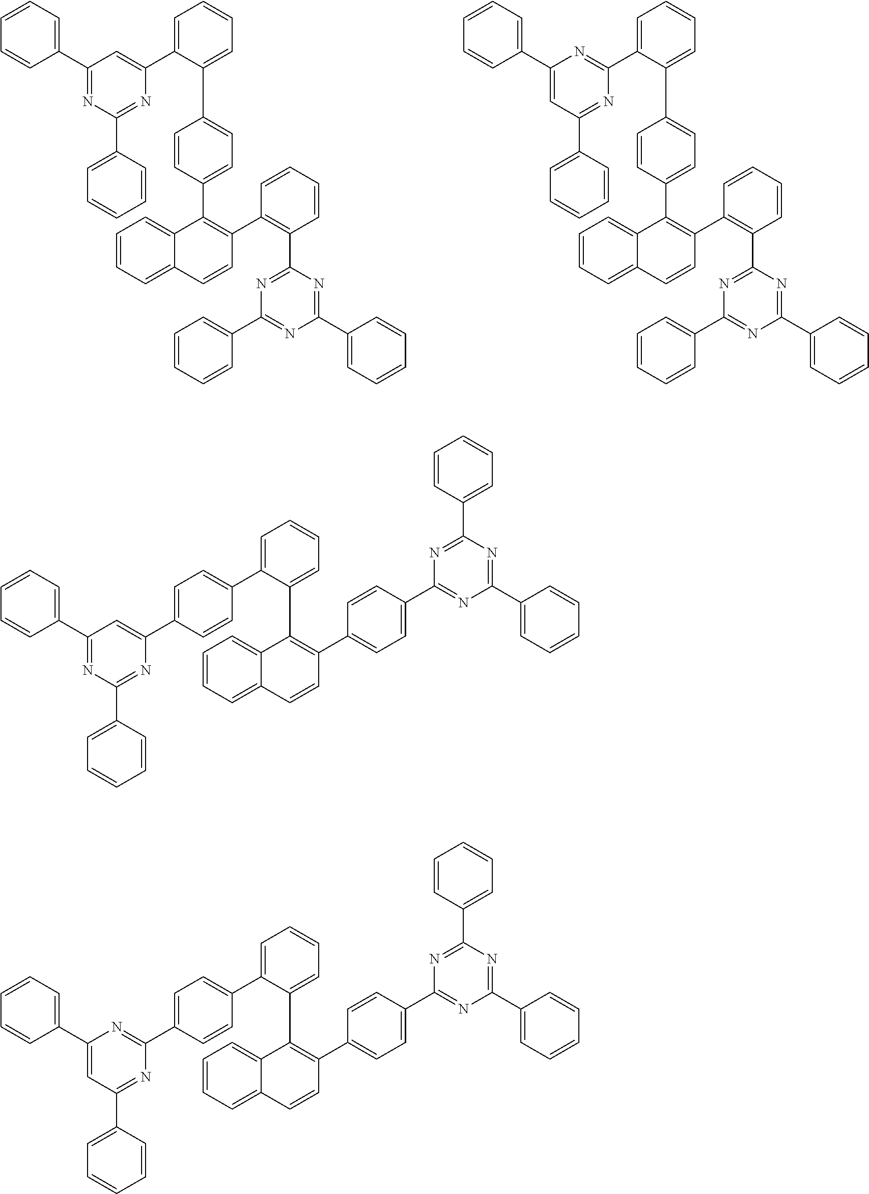 [SMILES: c1ccc(-c2cc(-c3ccccc3-c3ccc(-c4c(-c5ccccc5-c5nc(-c6ccccc6)nc(-c6ccccc6)n5)ccc5ccccc45)cc3)nc(-c3ccccc3)n2)cc1 ; c1ccc(-c2cc(-c3ccccc3)nc(-c3ccccc3-c3ccc(-c4c(-c5ccccc5-c5nc(-c6ccccc6)nc(-c6ccccc6)n5)ccc5ccccc45)cc3)n2)cc1 ; c1ccc(-c2cc(-c3ccc(-c4ccccc4-c4c(-c5ccc(-c6nc(-c7ccccc7)nc(-c7ccccc7)n6)cc5)ccc5ccccc45)cc3)nc(-c3ccccc3)n2)cc1 ; c1ccc(-c2cc(-c3ccccc3)nc(-c3ccc(-c4ccccc4-c4c(-c5ccc(-c6nc(-c7ccccc7)nc(-c7ccccc7)n6)cc5)ccc5ccccc45)cc3)n2)cc1]

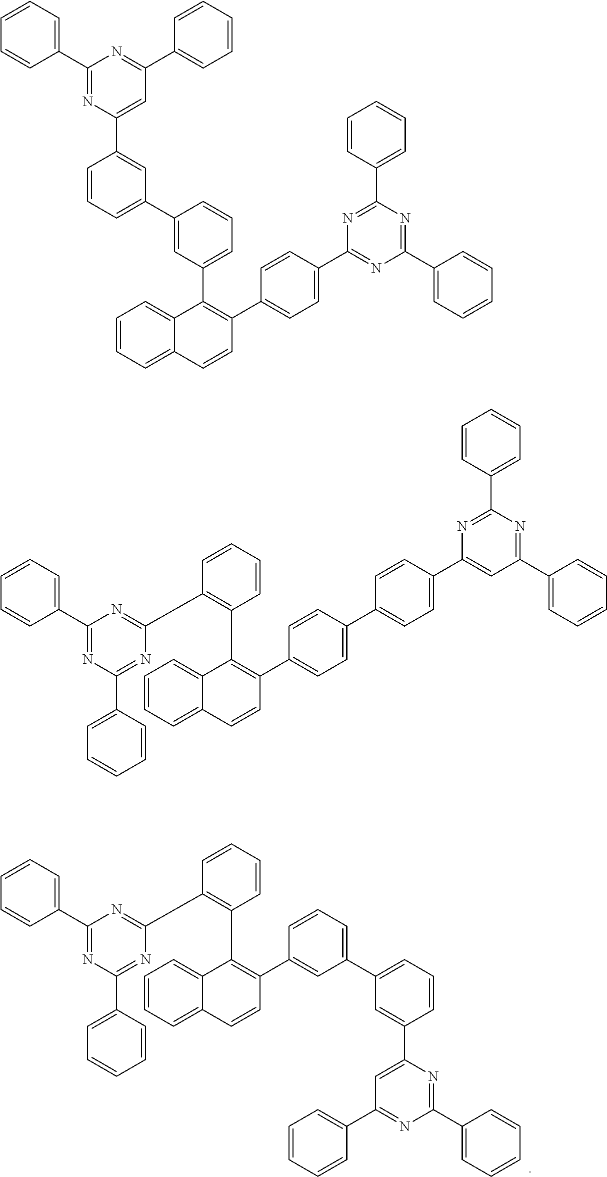 [SMILES: c1ccc(-c2cc(-c3cccc(-c4cccc(-c5c(-c6ccc(-c7nc(-c8ccccc8)nc(-c8ccccc8)n7)cc6)ccc6ccccc56)c4)c3)nc(-c3ccccc3)n2)cc1 ; c1ccc(-c2cc(-c3ccc(-c4ccc(-c5ccc6ccccc6c5-c5ccccc5-c5nc(-c6ccccc6)nc(-c6ccccc6)n5)cc4)cc3)nc(-c3ccccc3)n2)cc1 ; c1ccc(-c2cc(-c3cccc(-c4cccc(-c5ccc6ccccc6c5-c5ccccc5-c5nc(-c6ccccc6)nc(-c6ccccc6)n5)c4)c3)nc(-c3ccccc3)n2)cc1]

11. An organic light emitting device comprising:
an anode;
a cathode; and
an organic material layer having one or more layers provided between the anode and the cathode, wherein one or more layers of the organic material layer comprise the compound of Chemical Formula 1 of claim 1.

12. The organic light emitting device of claim 11, wherein the organic material layer comprises an electron transport layer, an electron injection layer, or an electron injection and transport layer, and
the electron transport layer, the electron injection layer, or the electron injection and transport layer comprises the compound of Chemical Formula 1.

13. The organic light emitting device of claim 12, wherein the electron transport layer, the electron injection layer, or the electron injection and transport layer further comprises an n-type dopant or organic metal compound.

14. The organic light emitting device of claim 13, wherein the compound of Chemical Formula 1 and the n-type dopant or organic metal compound are comprised at a weight ratio of 2:8 to 8:2.

15. The organic light emitting device of claim 11, wherein the organic material layer further comprises one or more layers of a hole injection layer, a hole transport layer, a hole injection and transport layer, an electron blocking layer, a light emitting layer, a hole blocking layer, an electron injection layer, an electron transport layer, and an electron injection and transport layer.

16. The organic light emitting device of claim 11, wherein the organic material layer comprises an electron injection and transport layer,
the electron injection and transport layer comprises the compound of Chemical Formula 1, and
the organic light emitting device further comprises one or two or more layers selected from the group consisting of a hole injection layer, a hole transport layer, an electron blocking layer, a light emitting layer, and a hole blocking layer.
